(19) United States
(12) Patent Application Publication
(10) Pub. No.: US 2023/0201380 A1
(43) Pub. Date: Jun. 29, 2023

(54) NON-INVASIVE AND LONGITUDINAL MONITORING OF MICROGLIAL ACTIVATION IN RAT BRAIN WITH SUPERMAGNETIC NANOPARTICLE ENHANCED MR IMAGING

(71) Applicant: UNM RAINFOREST INNOVATIONS, Alburquerque, NM (US)

(72) Inventors: Laurel Sillerud, Albuquerque, NM (US); Yi Yang, Albuquerque, NM (US)

(21) Appl. No.: 17/927,917

(22) PCT Filed: May 26, 2021
(86) PCT No.: PCT/US21/34313

§ 371 Date: Nov. 28, 2022

Related U.S. Application Data
(60) Provisional application No. 63/031,329, filed on May 28, 2020.

(51) Int. Cl. A61K 49/18

(52) U.S. Cl. CPC A61K 49/1887; A61K 49/1875

(57) ABSTRACT

After a stroke the temporal course of microglial/macrophage activation is biphasic. The initial phase promotes neuroinflammation, while the later phase aids neurovascular recovery. Therefore the dynamics of stroke-induced cerebral microglial/macrophage activation are of substantial interest. In one embodiment, the present invention is directed to the use of novel anti-Iba-1-targeted superparamagnetic FePt nanoparticles immunocelles in conjunction with magnetic resonance imaging (MRI) to measure the spatiotemporal course of the activation of microglia/macrophages in brain tissue at 7, 14, and 28 days post-stroke. Ischemic cerebral lesion areas are identified using T₂-weighted MR images. After injection of FePt nanoparticles as immunocelles, quantitative contrast changes in T₂*-weighted MR images showed that the nanoparticles were taken up solely in brain regions that coincided with areas of microglial/macrophage activation detected by post-mortem immunohistochemistry. There was observed good agreement between the locations of the Fe⁺-cells, as shown by Perl's staining for iron, and the Iba-V-microgiia/macrophages, The time course of nanoparticle uptake paralleled the changes of microglial/macrophage activation and phenotypes measured by immunohistochemistry over the four week period post-stroke. Maximum microglial/macrophage activation occurred seven days post-stroke for both measures, and the diminished activation found after two weeks continued to four weeks. The results evidence that nanoparticle-enhanced MRI constitute a novel approach for monitoring the dynamic development of neuroinflammation in living animals during the progression and treatment of stroke and neurodegenerative diseases. The implications and methods for diagnosis and monitoring therapy of stroke and other disease states and conditions are presented.

This application claims the benefit of priority from U.S. provisional application Ser. No. US63/031,329, filed May 28, 2020, the entire contents of said application being incorporated by reference herein.

### RELATED APPLICATIONS AND GRANT SUPPORT

This invention was made with government support under grant nos. R01 CA123194 and R21 NS091710 awarded by the National Institutes of Health (NIH). The government has certain rights in the invention.

### FIELD OF THE INVENTION

The present invention relates to nanoparticles, preferably FePt particles, which have been constructed to be useful in monitoring microglial/macrophage activation in the central nervous system and more particularly, the brain of a patient or subject. The nanoparticles according to the present invention may be used to provide enhanced magnetic resonance imaging of ischemic brain tissue in subjects in order to monitor microglial/macrophage activation to determine and characterize the initial inflammatory phase which occurs after stroke followed by the subsequent anti-inflammatory healing phase which follows. The monitoring and timing of these phases may be useful in diagnosing stroke and assisting therapeutic regimens for the treatment of stroke and related disease states and disorders.

### BACKGROUND AND OVERVIEW OF THE INVENTION

After a stroke the temporal course of microglial/macrophage activation is biphasic. The initial phase promotes neuroinflammation, while the later phase aids neurovascular recovery. Therefore the dynamics of stroke-induced cerebral microglial/macrophage activation are of substantial interest. Here, the inventors used novel anti-Iba-1-targeted superparamagnetic FePt nanoparticles in conjunction with magnetic resonance imaging (MRI) to measure the spatiotemporal course of the activation of microglia/macrophages in the living rat brain at 7, 14, and 28 days post-stroke. Ischemic cerebral lesion areas were identified using T₂-weighted MR images. After injection of the FePt nanoparticles, quantitative contrast changes in T₂*-weighted MR images showed that the nanoparticles were taken up solely in brain regions that coincided with areas of microglial/macrophage activation detected by post-mortem immunohistochemistry. In the present invention, the inventors observed good agreement between the locations of the Fe⁺-cells, as shown by Perl's staining for iron, and the Iba-1⁺-microglia/macrophages. The time course of nanoparticle uptake paralleled the changes of microglial/macrophage activation and phenotypes measured by immunohistochemistry over the four week period post-stroke. Maximum microglial/macrophage activation occurred seven days post-stroke for both measures, and the diminished activation found after two weeks continued to four weeks. Our results suggest that nanoparticle-enhanced MRI may constitute a novel approach for monitoring the dynamic development of neuroinflammation in living animals during the progression and treatment of stroke.

The neuroinflammation that is a prominent early response of the brain to cerebral ischemic stroke is an important contributor to post-stroke injury. However, later this neuroinflammatory response can also be beneficial in that it subsequently stimulates neurovascular remodeling^{1, 2}. Neuroinflammation in the ischemic brain results from the activation of microglia, the brain-resident macrophages, and from blood-borne macrophages from the circulation^{3, 4}. Microglia rapidly develop a pro-inflammatory phenotype in response to acute brain injury; meanwhile, activated microglia also present reparative and anti-inflammatory roles through a regulatory/homeostatic phenotype, which facilitates recovery after stroke^{3, 5-7}. Neuroinflammation accompanies these changes in the phenotypes of the microglia/macrophages after ischemic stroke, with one phenotype predominating over another in a time-dependent manner⁷⁻⁹. Dynamic analyses by ourselves and others have demonstrated that the location of the active microglia/macrophages with respect to the infarct in ischemic brain at different stages is a critical feature of these immune phenotypes^{7, 10-13}. We also found that in the peri-infarct areas adjacent to intact tissue a new population of active regulatory-phenotype microglia is involved in blood-brain-barrier (BBB) remodeling at 4 weeks after stroke¹⁴. Therefore, measurement of the temporal and spatial distribution of microglial/macrophage activation in vivo would provide insight into the development of neuroinflammation after cerebral ischemic stroke. This dynamic monitoring could be helpful for treatments targeting microglia/macrophages to find ways to suppress the deleterious effects of microglial activation without compromising neurovascular repair and remodeling^{1, 15}.

The non-invasive monitoring of microglial/macrophage activity in the living ischemic brain requires an in vivo imaging method that can specifically detect the microglia/macrophages. Magnetic resonance imaging (MRI) has long been applied to the diagnosis and surveillance of stroke in humans and animals because the resulting cerebral edema gives rise to T₂-weighted (T₂w) MR image hyperintensities. However, these images do not specifically report the activation status of the post-infarct microglia/macrophages. In order to accomplish this, magnetic materials targeted to a particular pathology are required that can be detected in the brain with MRI. Previously, we have developed a number of such specifically-targeted materials^{16, 17} and have successfully used MRI and both iron oxide-based, and novel iron-platinum (FePt)-based nanoparticles, for prostate cancer detection^{18, 19} and treatment²⁰. We have also shown that antibody-conjugated, superparamagnetic iron oxide nanoparticles (SPIONs), could be engineered to penetrate the BBB and to serve as an in vivo contrast agent for the specific MRI detection of amyloid-β plaques in transgenic Alzheimer's mice²¹.

However, until recently, the specific targeting of nanoparticles to microglia/macrophages in the ischemic brain had not been examined with MR Imaging. We have synthesized superparamagnetic FePt nanoparticles that reveal neuroinflammation through their antibody-mediated interaction with Iba-1, a 17 kDa protein specifically expressed in microglia/macrophages. Iba-1 expression by microglia/macrophages is significantly enhanced in the brain after ischemic stroke²². We recently showed that MR imaging enhanced with these Iba-1 antibody conjugated particles could serve as a sensitive, specific means to detect and quantify the neuroinflammation associated with Alzheimer's disease and its reduction after treatment with trans-stilbene NFκB inhibitors¹¹. We now report the application of these unique anti-Iba-1 conjugated FePt magnetic nanoparticles to the non-invasive, MR imaging of the time course and spatial distribution of microglial activation in the living rat brain after stroke.

As described in this application, the spontaneously-hypertensive rat (SHR) subjected to middle cerebral artery occlusion with reperfusion (MCAO/RP) was used as a model of cerebral ischemia. T₂w MR imaging was used to measure the anatomical location and extent of the infarcted region, while T₂*-weighted (T₂*w) MR imaging was employed to detect the distribution of the anti-Iba-1-conjugated FePt nanoparticle-labeled microglia/macrophages in rat brains. The MR imaging findings were compared with post-mortem histological analysis of the locations of the Fe⁺-cells and the active microglia/macrophages in the rat brains. We reconstructed the three-dimensional distribution of the activated microglia/macrophages in the whole brain using spatial measurements of the nanoparticle locations detected with T₂*w MR imaging. Here, we show that long-term spatiotemporal MRI monitoring of the labeled microglia/macrophages correlates with neuroinflammation occurring in living brain over time after an ischemic stroke.

### BRIEF DESCRIPTION OF THE INVENTION

After a stroke the temporal course of microglial/macrophage activation is biphasic. The initial phase promotes neuroinflammation (for a period of a few days often a week until about two weeks after a stroke), while the later phase aids neurovascular recovery (from about two weeks to about four weeks after stroke). Therefore the dynamics of stroke-induced cerebral microglial/macrophage activation are of substantial interest to provide insight into possible therapies and the timing of such. Here, the inventors used novel protein targeted (preferably anti-Iba-1-targeted) superparamagnetic FePt nanoparticles in conjunction with magnetic resonance imaging (MRI) to measure the spatiotemporal course of the activation of microglia/macrophages in the living rat brain at 7, 14, and 28 days post-stroke. Ischemic cerebral lesion areas were identified using T₂-weighted MR images. After injection of the FePt nanoparticles, quantitative contrast changes in T₂*-weighted MR images showed that the nanoparticles were taken up solely in brain regions that coincided with areas of microglial/macrophage activation detected by post-mortem immunohistochemistry. We observed good agreement between the locations of the Fe⁺-cells, as shown by Perl's staining for iron, and the Iba-1⁺-microglia/macrophages. The time course of nanoparticle uptake paralleled the changes of microglial/macrophage activation and phenotypes measured by immunohistochemistry over the four week period post-stroke. Maximum microglia/macrophage activation occurred seven days post-stroke for both measures, and the diminished activation (associated with healing) found after two weeks continued to four weeks. The initial results presented herein suggest that nanoparticle-enhanced MRI may constitute a novel approach for monitoring the dynamic development of neuroinflammation in living animals, especially humans during the progression and treatment of stroke, as well as other disease states and conditions where neuroinflammation is an important feature such as Alzheimer's disease, Huntington's Disease, Parkinson's disease, amyotrophic lateral sclerosis (ALS) and multiple sclerosis (MS).

In one embodiment, the invention provides a blood-brain barrier-permeable, PEGylated stealth immunomicelle comprising:

(a) a particulate core comprising a mixture of superparamagnetic particles (in preferred embodiments FePt nanoparticles, said core being encapsulated by a plurality of phospholipids comprising at least one pegylated phospholipid (preferably, stealth inducing as otherwise described herein), a phospholipid comprising conjugation functionalities (“a conjugatable phospholipid”, e.g., a crosslinkable or conjugatable phospholipid which may include a biotinylated phospholipid, among other conjugatable phospholipids, preferably functionalized/conjugatable pegylated phospholipids),
(b) a targeting antibody or peptide or other binding motif (e.g. a central nervous system targeting antibody or peptide such as anti-iba-1 or anti-GFAP (glial fibrillary acidic protein) which binds central nervous system proteins which are involved in neuroinflammatory processes associated with ischemia or other disease states and/or conditions as described herein. These are conjugated to the particulate core through an appropriate functionality of the conjugatable phospholipid (such that the antibody is preferably disposed at the surface of the immunomicelle); and
(c) optionally and preferably a blood-brain barrier-penetration ligand (e.g., polysorbate 80, and angiopep-1, among others) which is conjugated to the particulate core through a functionality of a conjugatable phospholipid which may be the same or different from the conjugatable phospholipid which binds the antibody, peptide or binding motif. In addition, the immunomicelle optionally comprises apoE2 to interact with the Low Density lipoprotein receptor and to assist in internalizing the nanoparticles into cells. The components of an embodiment of the PEGylated stealth immunomicelle of the present invention used in examples of the present application is set forth in Figure S6, Supplementary Table 1.

In one embodiment of the immunomicelle formulation:

(a) the superparamagnetic particles are superparamagnetic iron platinum particles (SIPP), superparamagnetic iron oxide nanoparticles (SPIONs) or superparamagnetic manganese oxide particles (SMIONs), preferably SIPPs;
(b) the targeting antibody or peptide is preferably a central nervous system antibody which binds to Iba-1 or to GFAP, preferably Iba-1;
(c) the blood-brain barrier-penetration component or ligand is polysorbate 80 or Angiopep-1 (both of which target the blood brain barrier).

In certain embodiments of the immunomicelle formulation:

(a) the stealth-inducing PEG phospholipid is 1,2-distearoyl-sn-glycero-3s-phosphoethanolamine-N-[methoxy(polyethylene glycol)-2000] ((DSPE-PEG);
(b) the conjugatable PEG phospholipid is 1,2-distearoyl-sn-glycero-3-phosphoethanolamine-N-[biotinyl(polyethylene glycol)-2000] ((DSPE-PEG-biotin); and
(c) a phospholipid which is 1,2-dipalmitoyl-sn-glycero-3-phosphocholine (16:0 PC (DPCC)).

In other embodiments of the immunomicelle formulation:

(a) the stealth-inducing PEG phospholipid is 1,2-distearoyl-sn-glycero-3s-phosphoethanolamine-N-[methoxy(polyethylene glycol)-2000] ((DSPE-PEG);
(b) the conjugatable PEG phospholipid is 1,2-distearoyl-sn-glycero-3-phosphoethanolamine-N-[biotinyl(polyethylene glycol)-2000] ((DSPE-PEG-biotin); and
(c) the crosslinking phospholipid is 1,2-bis(10,12-tricosadiynoyl)-sn-glycero-3-phosphoethanolamine ((Diyne-PE).

In embodiments, the immunomicelle comprises the components which are presented in figure S6 hereof.

In certain embodiments, the immunomicelle particulate core comprises an agent or combination of agents that can be used for both magnetic resonance imaging (MRI) and computed tomography (CT) to diagnose, image, and/or determine the stage of inflammation associated (e.g., activated microglial phase or neuroinflammation phase) or recovery (e.g. recovery phase with diminished or reduced microglial activity) with a stroke or central nervous system disorder (e.g. a neuroinflammatory disorder). The particulate core is preferably a superparamagnetic iron platinum nanoparticle (SIPP) that can be used for MRI and CT imaging and diagnosis, but is preferably used with MRI. Platinum has a high x-ray absorption coefficient of 6.95 cm²/g at 50 KeV, making the particles useful both as CT contrast agents and MRI contrast agents.

In certain embodiments, the encapsulated particulate cores described herein each have an average diameter of between about 10 nm and 150 nm, often about 15 to about 100 nm, about 20 to about 100 nm, about 25 to about 95 nm, more often between about 30 to about 70 nm, between about 40 to about 60 nm about 50-80, around 50 nm or about 80 nm. It is preferable that the encapsulated particulate cores have an average diameter of less than 100 nam. The paramagnetic core which is encapsulated using phospholipids often has an average diameter ranging from 2 to 90 nm, 5 to 85 nm, often 10 to 50 nm, often 10 to 35 nm, often 12 to 30 nm, more often 15 to 25 nm, most often less than 30 nm.

In other embodiments, the invention provides a pharmaceutical formulation comprising a plurality of the blood-brain barrier-permeable, PEGylated stealth immunomicelles as described herein, wherein the encapsulated particulate cores of each of said immunomicelles are preferably cross-linked, often by chemical conjugation or alternatively, by UV-light initiated polymerization.

In still other embodiments, the immunomicelle particulate core further comprises an agent or combination of agents for the treatment of non-cancer diseases or disorders (diseases other than cancer) associated with the central nervous system, such as agents for the treatment of stroke, or alternatively, agents for the treatment of Alzheimer's disease, Huntington's disease, Parkinson's disease, ALS or MS.

In still other embodiments of the present invention, the bioactive agents or drugs are modified with lipids to produce “lipid modified drugs”, for example, by conjugating C₄-C₁₈ lipids or fatty acids through, for example, ester or amide groups, among others to provide prodrug forms of the bioactive agents or drugs. In practice, the lipid modified drugs will not release the active drug until the lipid chains are cleaved off of the lipid modified drugs by esterases within the lysosome of the cells to be targeted by the drug.

In embodiments, the invention provides a method of diagnosing stroke in a subject in need (a subject who is at risk for a stroke or is likely to or believed to have had a stroke) comprising administering to the subject thereof a pharmaceutical formulation comprising a plurality of the blood-brain barrier-permeable, PEGylated stealth immunomicelles as described herein optionally in combination with a contrast agent (e.g., a gadolinium III containing contrast agent), obtaining an MRI or CT image of tissue in the patient after administration and comparing the MRI or CT image obtained from the subject to a standard (which may be an MRI or CT image of a normal subject or a subject having a stroke, identifiable standard measurements or data points from scans from normal patients or patients having a stroke or recovering from a stroke or other standard such as calibrating the uptake of particles by calculating the iron concentration per microglia as otherwise described herein, that can be compared to a MRI or CT image, to scan data or calculated particle uptake) in order to determine whether or not a patient is having a stroke or has had a stroke or other neurodegenerative disease and the stage of recovery that the patient is in (e.g. neuroinflammation consistent with activated microglia or neurovascular recovery). It is noted that a stroke will often be associated with increased activation of microglial cells within several days to a week after a stroke and that microglial activation typically lasts up to approximately two weeks after a stroke in a subject and is associated with neuroinflammation. This period will provide an opportunity for stroke treatment such as the administration of anti-inflammatory/immunomodulating agents, clot-dissolving (thrombolytic) agents such as tissue plasminogen activator (tPA), streptokinase or urokinase, blood pressure controlling agents and surgery (endovascular procedures, cerebral revascularization, embolectomy, thrombectomy, craniotomy, ventriculostomy, evacuation), among others. Typically, treatment for stroke will last from one week to eight weeks, often one week, two weeks, three weeks, two to four weeks, four weeks and four to eight weeks or in rare instances, longer than eight weeks.

In still other embodiments, the invention provides a method of simultaneously treating and imaging ischemia in a patient, comprising administering to a subject in need thereof a pharmaceutical formulation comprising a plurality of the blood-brain barrier-permeable, PEGylated stealth immunomicelles as described herein. In certain embodiments, methods of treatment of the invention are used to treat and image a stroke and/or other neuroinflammatory disorder as described herein. In embodiments, an MRI or CT image obtained at two or more times during therapy will indicate whether therapy is having a favorable impact of may need to be modified or terminated. In embodiments, the patient will be monitored at least three or more times (often up to 5 times depending on the duration of the treatment and severity of the stroke which is treated).

In other embodiments, the invention provides a method of simultaneously treating and imaging a primary ischemic event in a patient or subject comprising co-administering to a subject in need thereof a pharmaceutical formulation comprising a plurality of the blood-brain barrier-permeable, PEGylated stealth immunomicelles as described herein and one or more additional active ingredients or agents in treating ischemia. In embodiments, the treatment is monitored by imaging the patient at least two times during a period of therapy, at the start of therapy (which would be considered the control or standard) and at a later time after therapy has commenced to assess the effect of therapy in the patient. In embodiments, the therapy is terminated or modified because of a favorable or unfavorable result.

In still other embodiments, the invention provides a method of diagnosing the presence and/or progression in a subject of ischemia, comprising:

(a) administering an immunomicelle formulation of the invention to the subject;
(b) subjecting the subject to magnetic resonance imaging; and
(c) determining through MRI or CT contrast enhancement whether the subject suffers from ischemia and whether the ischemia is in a neuroinflammatory early phase or a later anti-inflammatory phase by comparing the resulting MRI or CT image from the subject with a control or standard (which may be a disease control/standard or a normal/healthy control/standard to which the subject's MRI image may be compared for diagnosis). It is noted that a control or standard may be an MRI image, a read-out of an MRI or other data such as change of control or other data, especially including iron uptake as described in more detail herein which may be readily used to compare the MRI of a subject or patient with either a normal/healthy patient or a patient with disease in varying states, as applicable.

Certain embodiments of these diagnostic methods further comprise measuring in a subject diagnosed with ischemia both the MRI or CT contrast enhancement of the volume at varying stages after ischemia. Other embodiments of the diagnostic methods determine the ability of the formulation to identify the phase of stroke in the subject and to determine the extent of activation of microglia in the subject.

In still other embodiments, the invention provides an in vivo method of diagnosing the presence or progression in a subject of a stroke and/or neuroinflammatory disorder associated therewith comprising:

(a) obtaining a sample from the subject;
(b) contacting the sample with a immunomicelle formulation of the invention and thereafter subjecting the sample to magnetic resonance imaging; and
(c) determining through MRI or CT contrast enhancement the stage of the stroke.

In still other embodiments, the invention provides a method of screening for a composition that arrests the progression of or facilitates treatment of a stroke comprising:

(a) contacting a cellular sample having a central nervous system phenotype or a neuroinflammatory disorder phenotype with a pharmaceutical formulation of the invention and thereafter subjecting the sample to magnetic resonance or computed tomography imaging; and
(b) thereafter evaluating one or more sample parameters selected from the group consisting of phenotype, MRI or CT contrast enhancement of the sample, and sample volume.

In other embodiments, the present invention is directed to a method for monitoring stroke treatment in a patient, the method comprising:

(a) administering a formulation of immunomicelles as described herein a subject who has suffered a stroke before the commencement of treatment of said subject to determine the level of microglial/macrophage activation;
(b) subjecting the subject to magnetic resonance imaging or CT scanning;
(c) determining the level of macrophage activation and/or neuroinflammation of the subject;
(d) commencing treatment of the subject;
(e) after a sufficient period of treatment of the subject, administering a formulation of immunomicelles as described herein to the subject to determine the level of microglial/macrophage activation;
(f) subjecting the subject to magnetic resonance imaging; and
(g) determining through MRI contrast enhancement or determination of iron content in microglia of said subject and the effect of treatment of said subject. In embodiments, the period of treatment is one week, two weeks, one to two weeks, one to three weeks, one to four weeks, one to five weeks, one to six weeks and four to eight weeks, or occasionally longer.

Thus, the invention in certain embodiments provides novel immunomicelles that are blood-brain barrier permeable and that are specifically targeted to markers (such as Iba-1) associated with microglial macrophage activation during ischemia to cells for both imaging and therapy, and that are also useful in the diagnosis and treatment of neuroinflammatory disorders such as Alzheimer's Disease, Huntington's disease, Parkinson's disease, ALS or MS.

As described herein, the inventors have synthesized immunomicelles comprising superparamagnetic iron platinum particles (SIPP), superparamagnetic iron oxide nanoparticles (SPIONs) or superparamagnetic manganese oxide particles (SMIONs) for use as diagnostics for use in identifying microglial/macrophage activation therapeutics in the diagnosis and treatment of ischemia and for potential use as in vivo imaging agents in detecting primary central nervous system activation of microglial/macrophages during ischemia or other neuroinflammatory disorders.

In one embodiment, the invention provides novel superparamagnetic iron platinum nanoparticles (SIPPs: Taylor et al., 2011; 2012) conjugated to anti-iba-1 antibodies that recognize activated microglia and use these nanoparticles to measure and treat stroke.

In certain alternative embodiments of the invention relating to diagnosis and assessment of treatment of patients at risk for or diagnosed with stroke, nanoparticles of superparamagnetic nanoparticles, preferably iron platinum nanoparticles (SIPPS) or iron oxide nanoparticles (SPIONS) which may be polydisperse or monodisperse (i.e., particles are all or nearly all the same size) are conjugated to an antibody (e.g. monoclonal or polyclonal) which binds Iba-1. The superparamagnetic nanoparticles may also be magnetite (SiMAG-TCL (Chemicell, Berlin, Germany)) which are conjugated with a conjugating agent such as N-hydroxysulfosuccinimide (Sulfa-NHS) and 1-Ethyl-3-[3-dimethylaminopropyl]carbodiimide hydrochloride (EDC), among others as described herein, coupled to an anti-iba-1 or anti-GFAP (glial fibrillary acid protein) polyclonal or monoclonal antibody (preferably, an anti-iba-1 monoclonal antibody, and used in combination with magnetic resonance imaging to assess in a subject levels of macrophage activation to determine the state of ischemia in the patient with MRI and comparing the measurements obtained with a standard) to diagnose stroke and/or assess the progress of treatment of disease in a patient, among other methods.

Compositions according to the present invention may be formulated in pharmaceutical dosage form (often as an intravenous dosage form) and delivered to the patient or subject to be diagnosed and/or treated. Diagnosis of stroke and/or monitoring of therapy for stroke occurs by magnetic resonance imaging.

As described in detail herein, studies in spontaneously-hypertensive rat (SHR) subjected to middle cerebral artery occlusion with reperfusion (MCAO/RP) demonstrate the ability of the novel protein-conjugated SIPPS immunomicelles to penetrate the blood-brain barrier and to act as a contrast agent for MR imaging of microglial/macrophage activation in order to diagnose stroke and/or assess the progress of disease treatment.

These and other aspects of the invention are described further in the detailed description of the invention as described herein below.

### BRIEF DESCRIPTION OF THE FIGURES

FIG. 1 shows a summary of the rat experimental groups, and regions of interest (ROI) used for MRI image intensity measurements. A. An outline of the experimental protocol showing the timing of the MRI scans, the injections of the SM80s, and the post-mortem histological analysis at various reperfusion (RP) times after MCAO. Groups 1, 3, and 4 received one injection of Iba-1-targeted SM80s (Iba-1-MS80s) and one MRI scan at 1, 2, and 4 weeks, respectively. Group 5 received three injections of Iba-1-targeted SM80s and three MRI scans at 1, 2, and 4 weeks. Group 2 received one injection of non-Iba-1-targeted, control SM80s (MS80s) and one MRI scan at 1 week. Sham rats received three injections of Iba-1-SM80s and three MRI scans at 1, 2, and 4 weeks. The time of various procedures is indicated as MCAO: a 90 minute middle cerebral artery occlusion; RP (↓): the time at which reperfusion was begun. FePt (↓): nanoparticle injection time-point. MRI (↓): MRI scan time-point. His (↓): post-mortem histological analysis time-point. B. A representative slice from T₂-weighted image of a Group 1 rat brain taken 7 days after MCAO/RP showing the ROIs used for the measurement of the signal intensities in the ipsilateral and contralateral portions of the brain. Within each hemisphere, three regions, including the cortex (1, 2) and striatum (3), were measured. MCAO was performed on the right hemisphere (R, ischemic), while the left hemisphere served as a nonischemic (L, nonischemic) control region. Note the hyperintensity of the right cortex compared with the left due to edema.

FIG. 2 shows active microglia shown by MRI and histological analysis in the brain of a rat (Group 1) at 1 week after MCAO/RP. A. MR T₂w and T₂*w images taken before (pre-injection) of Iba1-SM80s. The anatomic T₂w image shows the lesion as a right-sided hyperintense region. A T₂*w gradient-recalled echo image of the same slice. B. MR T₂w and T₂*w images taken 24 hours post-injection (after-injection) of Iba1-SM80s. A T₂w image of the brain of this rat showing the same right-sided hyperintense region. A T₂*w image showing the location of the magnetic nanoparticles as punctate hypointensities. CTX: cortex; Str: striatum. C. Intensity profile of the T₂*w MR image from (B, Left) taken along the indicated white line in the T₂*w post-injection image. Note that the intensity is lower in the ischemic hemisphere (arrow) compared to the nonischemic one. Ven: Ventricle. D. A post-mortem formalin-fixed brain slice from the same rat shows that the lesion area in the ischemic hemisphere (arrow) coincides with that seen in the T₂w image in (A, Left). E. A brain section immunofluorescently stained for Iba-1 (green) shows that the distribution of active microglia in the ischemic hemisphere of this brain matched the areas of hyperintensity seen in the T₂w image (A, Left), F. The distribution of Fe⁺-microglia determined using Perl's Prussian blue staining in the non-ischemic hemisphere and in the ischemic hemisphere of the same brain as in (A). Note that Fe⁺-microglia only appear in the brain tissue from the ischemic region. V: vessel. Scale bars=100 μm.

FIG. 3 show MR imaging and histological analysis which evidence that Iba1-SM80s produce specific imaging changes compared with control, non-Iba1-targeted-SM80s (SM80s) in ischemic hemispheres at 1 week after MCAO/RP. A. T₂w and T₂*w MR images of the brains from rats injected with either Iba1-SM80s (Left column, Group 1) or SM80s (Right column, Group 2). The post-injection, T₂w images (Top row) show that the spatial extant and size of the cortical and striatal edema in the infarcted regions was similar in both groups, but that injection of only the full, Iba1-SAM80s produced punctate hypointensities in the cortex ((Top, Left) compared with the smooth appearance in the rats injected with the control SM80s. The arrow indicates the ischemic hemisphere. The pre-injection T₂*w MR images (Middle row) show a smooth cortex and striatum. The post-injection T₂*w MR images (Bottom row) show the SM80s as punctate hypointense regions in only the image from the rat injected with Iba1-SM80s (Bottom, Left), while no such effect is seen when control SM80s were used (Bottom, Right). B. The effect of magnetic nanoparticle injection on the quantitative contrast of the T₂*w MR images in rat brains obtained pre- and after nanoparticle injections. The intensities of the 3 ipsilateral regions of interest (in the ischemic hemisphere) and those of the 3 contralateral of interest (in the nonischemic hemisphere) (see FIG. 1B) were measured and used to compute the Right (R)/Left (L) contrast as C=(Rᵢ−Lᵢ)/Lᵢ, i∈{1,2,3}. This contrast decreased significantly (P=0.001) after the injection of the Iba1-SM80s (B, Left), while no significant contrast change was observed after the injection of the control SM80s (B, Right; P=0.16). (C.) Post-injection rat brain T₂*w MR image contrast obtained after injections with either Iba1-SM80s or SM80s. There was a significant difference (P=0.02) in contrast obtained after injection of the Iba1-SM80s (C, Left) compared with the control SM80s. D. Quantitative contrast changes (ΔC) between T₂*w MR images obtained in rat brains before (Cₚ) and after-injection (Cₐ) with either Iba1-SM80s or control SM80s. Sham rats were injected with Iba1-SM80s and there was no contrast change, ΔC=0.009. The brains form Group 1 rats injected with Iba1-SM80s showed a significantly different negative contrast change of ΔC=−0.087. Injection of control SM80s into rats produced only a slightly negative ΔC=−0.036. **p<0.01 vs. sham and SM80s groups. ^{#}p<0.05 vs. sham group. N=6 rats in the sham group, n=11 in the Iba1-SM80s group, and n=10 in the control SM80s group. E. Perl's Prussian Blue staining for Fe⁺-microglial cells in the ischemic hemisphere in the brains of rats injected with control SM80s showing no Fe⁺-cells in the cortex (CTX) only a few (˜6) in the area of the striatum (Str). Scale bars=50 μm.

FIG. 4 show a comparison of MR images obtained after single vs. multiple injections of the Iba-1-MS80s shows that image intensity perturbations decay within a few days so that the SM80s do not accumulate in the brain tissue. A. T₂w and T₂*w MR images of the sham rat brains obtained 24 hours after the injection of Iba1-SM80s at 1, 2, and 4 weeks (WK). These animals received 3 injections of Iba1-SM80s. B. T₂w and T₂*w MR images of the brains of Group 5 rats obtained 24 hours after the injection of Iba1-SM80s at 1, 2, and 4 weeks after MCAO/RP. These animals received 3 injections of Iba1-SM80s. Substantial punctate hypointensities in the T₂*w images were found in the whole ischemic area at one week, which were also seen in the regions surrounding the core infarct areas (bright high intensity) at 2 and 4 weeks. C. T₂w and T₂*w MR images obtained 24 hours after-injection of Iba1-SM80s into rats at 1 week (Group 1), 2 week (Group 3), and 4 week (Group 4) after MCAO/RP. These animals received single injection of Iba1-SM80s at 1, 2, and 4 weeks, respectively. Here the hypointensities in the T₂*w images were similar to that observed in (B). D. Perl's Prussian Blue staining for Fe⁺-microglia in the sham or in the MCAO/RP rats from (A and B) at 4 weeks, after receiving 3 injections. Photos were taken from the cortex (CTX) and striatum (Str) areas shown as arrowheads in (A and B). Scale bars=100 μm. Note the lack of significant blue staining in the tissue from sham rats, which is in contrast to the residual blue staining seen in the 4-week MCAP/RP group. E. Quantitative contrast (C) in the T₂w MR images of the rat brains taken 24 hours after either single or multiple injections of Iba-1-SM80s at 1, 2, and 4 weeks after MCAO/RP, m: multiple-injection groups (sham group and Group 5). s: single-injection groups (Groups 1, 3, and 4). *p<0.01 vs. 1-injection MCAO/RP groups at 1 week (1W), ***p<0.001 vs. 1-injection and 2-injectors MCAO/RP groups at 2 weeks (2W), ****p<0.0001 vs. 1-injection and 3-injection MCAO/RP group at 4 weeks (4W) F. Comparison of the contrast changes (ΔC=Cₐ−Cₚ) of T2*w MR images between rats with three serial injections and rats with single injection, at 1, 2, and 4 weeks after MCAO/RP. **p<0.01 vs. 1-injection MCAO/RP groups at 1 week, *p<0.05 vs. 1-injection and 2-injectors MCAO/RP groups at 2 weeks. n=6 in 3-injection sham group; n=11, 9, and 10 in 1-injection MCAO/RP groups; n=8 in 3-injection MCAO/RP group.

FIG. 5 shows longitudinal monitoring of microglial activation with T₂w and T₂*w MR imaging and histochemistry. A. The changes of the distribution of the hypointense regions in the T₂*w images reflected the distribution of active microglia/macrophages by Iba-1 staining in the same ischemic hemispheres over 4 weeks after MCAO/RP. The left column of T₂*w MR images shows the appearance of the rat brain 24 hours after Iba-1-SM80 injections in sham rats and rats subjected to MCAO/RP for 1, 2, and 4 weeks. In the right column, the brains from the same animals were removed after MRI and stained for Iba-1 to reveal the location and density of activated microglia. B. Iba-1 and Perl's Prussian blue staining on adjacent sections of each rat from (A). The changes of morphology and density of Fe⁺-cells in the ischemic hemispheres reflected the changes of active microglia/macrophages stained by Iba-1. Note that the marked right-sided edema-related hyperintensities in the T₂*w images at 2 and 4 weeks were observed by histology as fluid-filled voids, due to cell death and tissue atrophy in the core infarcts. Scale bar=50 μm. The inserts show higher magnification views of the microglia/'macrophages stained for Iba-1 and Perl's Prussian blue C. The time dependence of the quantitative contrast (Cₐ) in T₂w and T₂*w MR images from sham or MCAO/RP rat brains following both single and three injections of Iba1-SM80s at weeks 1, 2, and 4 after MCAO/RP. The T₂w contrast in the MCAO/RP groups were significantly increased compared to the sham group at all time points due to edema formation. There was no significant difference of T₂w contrasts between the Iba-1-MS80s rats and the control MS80s rats at 1 week. *p<0.05 vs. 1W/RP of rats with Iba1-SM80 s or control SM80s; ****p<0.0001 vs. 2W and 4W of sham rats. The quantitative contrast measured in T₂*w MR images was independent of time for the sham rats. Significantly different was seen in the 1W/RP group injected Iba1-SM80s compared to the sham, control SM80s, 2 and 4 weeks MCAO/RP groups. *p<0.05 vs. 1W of sham and 1W/RP of control SM80s groups, ^{##}p<0.01 vs. 2W/RP and 4W/RP of rats with Iba1-SM80s. Injection of control SM80s at 1 week did not produce significant different contrast from the sham group. The time dependence of the contrast changes (ΔC) in T₂w and T2*w MR images obtained from sham or MCAO/RP rats following single and three injections of Iba1-SM80s at weeks 1, 2, and 4 after MCAO/RP. The most significantly negative ΔC was seen in the T₂*w images at 1 week for the MCAO/RP rats injected with Iba1-SM80s; this value decreased at 2 and 4 weeks. ***p<0.001 and **p<0.01 vs. MCAO/RP groups at 1 week or 2 weeks. ^{#}p<0.05 vs. sham group. ^{##}p<0.01 vs. control SM80s group. N=6 in sham group; n=19 in MCAO/RP group at 1 week; n=17 in MCAO/RP group at 2 week; n=18 in MCAO/RP group at 4 week; and n=10 in control SM80s group at 1 week.

FIG. 6 shows three-dimensional (3D) mapping of the Iba-1-SM80s within the brain parenchyma A. T₂*w MR images of the brain of a Group 1 rat injected with Iba-1-SM80s 7 days after MCAO/RP. The arrows show the ischemia produced by MCAO. Images were obtained from sixteen slices whose locations in the brain ranged from ˜Bregma+1.70 to −7.64 mm. B. The Z-score distribution of the Iba1-SM80s in 3-dimensions within the brain where the location corresponded to the punctate regions of low signal intensity and the radius and color of the plotted spheres corresponded to the Z-score (See Methods for details of the mapping used in Mathematical). Shown are three different views of the same data set, but individually rotated for clarity. Note that the Iba1-SM80s cluster in the region of the infarct and nowhere else within the brain.

FIG. 7 shows inflammatory cytokines expressed by active microglia/macrophage in the infarct (inf) areas bordering with the peri-infarct (peri-inf) areas at 1, 2, and 4 weeks (W) after stroke and reperfusion. A. Double immunostaining shows the expression of YM1 in active microglia (OX-42). DAPI was used to show the nuclei. Scale bar=50 μm. Statistical Li's ICQ values for co-localization of YM1 with OX-42 in sham and ischemic hemispheres. *P<0.05 vs. 2 week, **P<0.01 vs 4 weeks. B. Double immunostaining shows expression of TNF-α and IL-1β in active microglia (Iba-1). Statistical Li's ICQ values demonstrate the quantification of co-localization of TNF-α and IL-1β with Iba-1 in sham and ischemic hemispheres. TNT-α: **P<0.05 vs. 2 weeks, ****P<0.0001 vs. 1 week, ***p<0.001 vs. 1 week. IL-1β: **P<0.01 vs. 2 and 4 weeks, ***p<0.001 vs. 1 week. C. Double immunostaining shows expression of TGF-β and IL-10 in active microglia (Iba-1). Statistical Li's ICQ values demonstrate the quantification of co-localization of TGF-β and IL-10 with Iba-1 in sham and ischemic hemispheres. TGF-β: **P<0.01 vs. 2 week, ***p<0.001 vs. 4 weeks; ^{##}p<0.01 vs. 2 weeks, ^{###}p<0.001 vs. 4 weeks. IL-10: *P<0.05 vs. 2 and 4 weeks, ***p<0.001 vs. 2 and 4 weeks. n=3 in sham groups, n=6 in groups of 1, 2, 4 weeks reperfusion (RP).

Figure S1 shows the asymmetry of the intensity seen between the left and right brain hemispheres in the T₂w and T₂*w MR images. (A) T₂w and T₂^{*}w MR images of sham rat brains after Iba1-SM80 injection at 1, 2 or 4 weeks (WK) after sham surgery. Note that in these control brains there is an asymmetry of the intensity between the Left and Right brain hemispheres due to the inherent radiofrequency inhomogeneity of the surface coil used to obtain the images. (B) Graphs comparing the intensities of T₂w and T₂*w MR images between left and right hemispheres scanned after Iba1-SM80s injection at 1, 2 and 4 weeks (n=6). The measured mean image intensity ratio of Left to Right was found to be 1.20, and this value was used to correct all the contrast measurements.

Figure S2 shows the brain contrast difference ΔC after the injection of SM80s as a function of the [Fe].

Figure S3 shows the analysis and quantification for co-localization of cytokine (IL-1β) and Iba-1 in the microglia/macrophages with the colocalization plugin Coloc2 program in Fiji-ImageJ in brain sections from sham or MCAO/RP rats. A. Double immunostaining of IL-1β and Iba-1 and two-dimensional histogram and scatterplot generated by plugin Coloc2 in sham rat brain. The ICQ value of 0.144 indicates very weak colocalizaiton. B. Double immunostaining of IL-1β and Iba-1 and two-dimensional histogram and scatterplot generated by plugin Coloc2 in 4 week-MCAO/RP rat brain. Here, the ICQ value of 0.427 shows strong colocalization. The two-dimensional histogram and scatterplot display the correlation of the pixel intensities, over all pixels and voxels in the images with different Li's ICQ (Intensity Correlation Quotient) values. The ICQ is defined as the ratio of positive products divided by the overall products minus 0.5. As a consequence, the ICQ varies from 0.5 (co-localization) to −0.5 (exclusion) while random staining and images impeded by noise will give a value close to zero (Random staining: ICQ˜0; Segregated staining: 0>ICQ≥−0.5; Dependent staining: 0<ICQ≤+0.5)^{9, 10}.

Figure S4 shows the effect of nanoparticle injection on the intensities of the nonischemic (L) and ischemic (R) hemispheres, 1 week after MCAO/RP, in T₂*w MR images of rat brains obtained 24 hours after injection. aft: after-injection. (A) Comparison of the intensities after injection with Iba1-SM80s. The Left and Right intensities are significantly different, P=0.0033; n=10. (B) Comparison of the intensities after injection with control SM80s. The Left and Right intensities are not significantly different, P=0.1197; n=11.

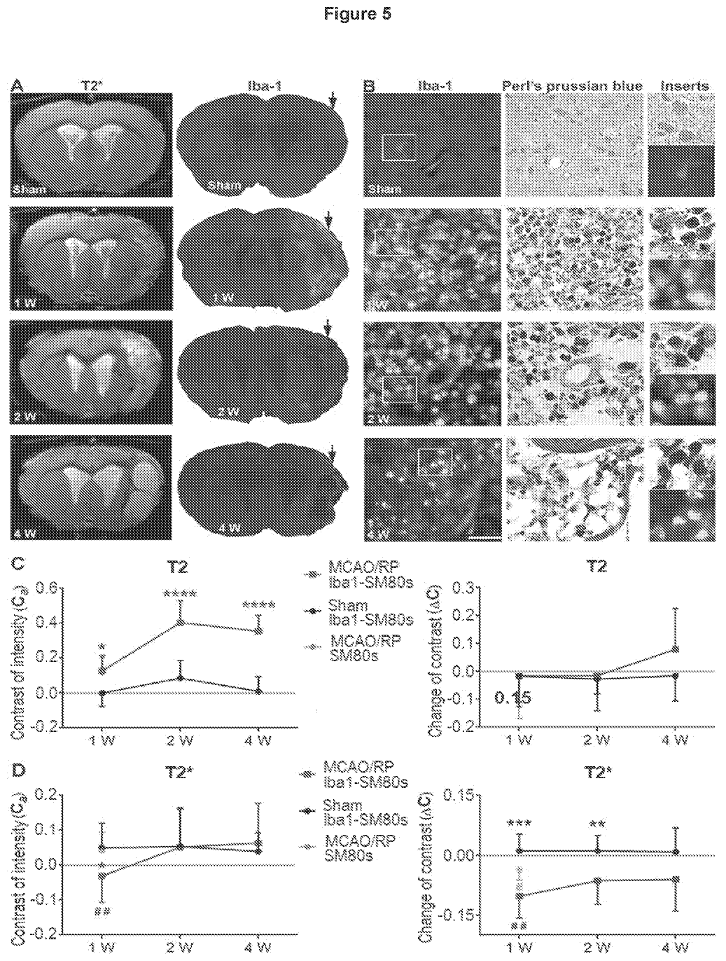

Figure S5 shows that the Iba-1 SM80s do not accumulate in the brain after multiple injections. This figure compares the effect of single or multiple injections of Iba-1-SM80s into sham and MCAO/RP rats on the contrast (Cₐ) in T₂*w MR images taken 24 hours after-injection. (A) Contrast after a single injection at 1 week. The central plot is for the group of MCAO/RP rats that only received a total of one injection (*p<0.01 Sham vs. 1-injection MCAO/RP groups at 1 week. m: multiple-injection group. s: single-injection group. (B) Contrast at 2 weeks with either the second injection (2-inj) or the first injection (1-inj) at 2 weeks (The central 2WL/RP). (C) Contrast at 4 weeks with either the third injections (3-inj) or a single injection at 4 weeks (1-inj) (n=6 in 3-injection sham group; n=11, 9, and 10 in 1-injection MCAO/RP groups; n=8 in 3-injection MCAO/RP group). The data from these individual groups were pooled and used to generate the Right-hand plot in FIG. 5C.

Figure S6 shows Table 1 which is directed to components used for the construction of SIPP micelles used in the examples which are injected into rats weighing 280 g each.

### DETAILED DESCRIPTION OF THE INVENTION

The following terms are used throughout, the specification to describe the present invention. Where a term is not given a specific definition herein, that term is to be given the same meaning as understood by those of ordinary skill in the art. The definitions given to the disease states or conditions which may be treated using one or more of the compounds according to the present invention are those which are generally known in the art.

It is noted that, as used in this specification and the appended claims, the singular forms “a,” “an,” and “the,” include plural referents unless expressly and unequivocally limited to one referent. Thus, for example, reference to “a compound” includes two or more different compounds. As used herein, the term “include” and its grammatical variants are intended to be non-limiting, such that recitation of items in a list is not to the exclusion of other like items that can be substituted or other items that can be added to the listed items.

As described in United States Patent Application Document No. 20110263833 (“Compositions for Isolating a Target Analyte from a Heterogeneous Sample”), the complete contents of which are hereby incorporated by reference, magnetic particles can be permanently magnetizable, or ferromagnetic, or they may demonstrate bulk magnetic behavior only when subjected to a magnetic field. Magnetic particles that exhibit bulk magnetic behavior only when subjected to a magnetic field are “magnetically responsive particles” or are also characterized as “superparamagnetic”. Materials exhibiting bulk ferromagnetic properties, e.g., magnetic iron oxide, may be characterized as superparamagnetic when provided in crystals of about 30 nm or less in diameter.

The superparamagnetic particles used in the instant invention include all of the superparamagnetic particles and superparamagnetic nanoparticles and mixtures thereof described in United States Patent Application Document No. 20110263833. Preferably, a “superparamagnetic particle” is a superparamagnetic iron platinum particle (SIPP) or a superparamagnetic iron oxide nanoparticle (SPION). SIPP particles are preferred for use in the present invention.

SIPPS/SPIONS may be polydisperse or monodisperse (i.e., particles are all or nearly all the same size) which are conjugated to an antibody which hinds Iba-1. In certain embodiments, SPIONs comprise magnetite (SiMAG-TCL (Chemicell, Berlin, Germany) and are conjugated with a conjugating agent such as N-hydroxysulfosuccinimide (Sulfo-NHS) and 1-Ethyl-3[dimethylaminopropyl]carbodiimide hydrochloride (EDC) and coupled to anti-iba-1 or GFAP polyclonal or monoclonal antibody (preferably, an anti-iba-1 monoclonal antibody, which are then used in combination with magnetic resonance imaging to assess in a subject levels of microglial/macrophage activation associated with ischemia (by measuring the amount of Iba-1 or GFAP protein in a subject with MRI and comparing the measurements obtained with a standard) to diagnose the stage of ischemic in the subject and/or assess the progress of treatment of disease in a patient, among other methods. Once prepared, the composition according to the present invention may be formulated in pharmaceutical dosage foam (often as an intravenous dosage form) and delivered to the patient or subject to be diagnosed. Diagnosis occurs by magnetic resonance imaging or computed tomography, most often by MRI.

In certain embodiments, SIPPS/SPIONS comprise paramagnetic nanoparticles, generally approximately 1-3 nanometers (nm) to about 100 nm, about 5nm to about 100 nm, about 9-10 nm to about 50 nm, about 5 nm to about 25 nm in diameter, often less than 30 nm which comprise a paramagnetic iron material, preferably ferrous platinum (FePt), ferric oxide (Fe₂O₃), ferrous oxide (FeO) or ferroferric oxide (Fe₃O₄) which is coated with a polymeric coating which is preferably hydrophilic. Preferably, the SIPPS/SPIONS (comprising the iron platinum or iron oxide spheres as well as the polymeric coating) are 1-, 20, 30, 40, 50 or 100 nm in hydrodynamic diameter. The polymeric material which coats the particles may be a hydrophilic polymer such as chitosan, dextran (or any one or more of its pharmaceutically acceptable derivatives such as dextran sulfate and carboxymethyl dextran, among others), starch (or any one or more of its pharmaceutically acceptable derivates such as hydroxyethyl starch, hydroxypropyl starch, cationic starch, hydroxymethylstarch and carboxymethylstarch, among others) or a lipid or most preferably a phospholipid (e.g., phosphatidylcholine) or phospholipid mixture to protect against aggregation or clumping of the nanoparticles. In preferred embodiments, the polymeric coating contains PEG of approximately 1,000-5,000 molecular weight. In embodiments PEG is conjugated to a phospholipid and used in a phospholipid mixture for coating the paramagnetic core nanoparticles. The hydrodynamic diameter consists of the thickness of the core iron oxide particle as well as the external polymer coating.

In certain embodiments, the invention provides contrast agents comprising a population of nanoparticles effective to diagnose the stage of macrophage activation in stroke patients.

In one embodiment of an immunomicelle of the invention:

(a) the superparamagnetic particles are either superparamagnetic iron platinum particles (SIPP) or superparamagnetic iron oxide nanoparticles (SPION), preferably SIPP;
(b) an active ingredient that is useful in the treatment of stroke;
(c) an antibody that targets Iba-1 or GFAP, and
(d) the blood-brain barrier-penetration ligand is Angiopep-1 or is polysorbate 80 or ApoE2.

The phospholipid components of the stealth-inducing PEG phospholipid and conjugated phospholipid, often a PEG phospholipid (which may be used in further conjugation with an antibody) include, but are not limited to, the phospholipid components contained within or identified as the following compositions: PEG-polyethylene glycol)-derivatized distearoylphosphatidylethanolamine (PEG-DSPE), polyethylene glycol)-derivatized ceramides (PEG-CER), hydrogenated soy phosphatidylcholine (HSPC), egg phosphatidylcholine (EPC), phosphatidyl ethanolamine (PE), phosphatidyl glycerol (PG), phosphatidyl insitol (PI), monosialogangolioside, spingomyelin (SPM), distearoylphosphatidylcholine (DSPC), dimyristoylphosphatidylcholine (DMPC), and dimyristoylphosphatidylglycerol (DMPG), among others.

“Conjugated phospholipids” or “conjugatable phospholipids” include any phospholipid, including pegylated phospholipids as otherwise described herein which are conjugated to antibodies and/or peptides (or other targeting motifs) or contain conjugatable moieties which may be used to conjugate antibodies and/or peptides (or other targeting motifs). Conjugation of a phospholipid to an antibody or peptide (or other targeting motif) occurs through any method known in the art, including amine to amine conjugation, amine to sulfhydryl conjugation, carboxyl to amine conjugation, sulfhydryl to hydroxyl conjugation, sulfhydryl to sulfhydryl conjugation, click chemistry, imidoesters, NHS-esters, photoreactive conjugation, the streptavidin/biotin interaction, etc. These methods of conjugation are well known in the art and may be readily adapted for conjugating antibodies and targeting peptides (or other binding motifs) to any phospholipid, including pegylated phospholipids which may be used in the present invention.

Exemplary conjugatable phospholipids for use in the present invention include, for example:
1,2-Dioleoyl Phosphatidylethanolamine (DOPE);
1,2-Dipalmitipyl-sn-Glycero-3-Phosphoethanolamine (DPPE);
16:0 PE MCC (1,2-dipalmitoyl-sn-glycero-3-phosphoethanolamine-N-[4-(p-maleimidomethyl)cyclohexane-carboxamide];
18:1 PE MCC (1,2-dioleoyl-sn-glycero-3-phosphoethanolamine-N-[4-(p-maleimidomethyl)cyclohexane-carboxamide];
18:0 aminoethyl PC 1,2-distearoyl-sn-glycero-3-phosphocholine N-aminoethyl);
18:1 aminoethyl PC 1,2-dioleoyl-sn-glycero-3-phosphocholine (N-aminoethyl);
18:1 MPB PE 1,2-dioleoyl-sn-glycero-3-phosphoethanolamine-N-[4-(p-maleimidophenyl)butyramide];
16:0 MPB PE 1,2-dipalmitoyl-sn-glycero-3-phosphoethanolamine-N-[4-(p-maleimidophenyl)butyramide];
18:1 Caproylamine PE 1,2-dioleoyl-sn-glycero-3-phosphoethanolamine-N-(hexanoylamine);
16:0 Caproylamine PE 1,2-Dipalmitoyl-sn-Glycero-3-Phosphoethanolamine-N-(hexanoylamine);
16:0 Dodecanylamine PE 1,2-dipalmitoyl-sn-glycero-3-phosphoethanolamine-N-(dodecanylamine);
18:1 Dodecanylamine PE 1,2-dioleoyl-sn-glycero-3-phosphoethanolamine-N-(dodecanylamine);
16:0 Ptd Thioethanol 1,2-Dipalmitoyl-sn-Glycero-3-Phosphothioethanol;
18:1 PDP PE 1,2-dioleoyl-sn-glycero-3-phosphoethanolamine-N-[3-(2-pyridyldithio)propionate];
16:0 PDP PE 1,2-dipalmitoyl-sn-glycero-3-phosphoethanolamine-N-[3-(2-pyridyldithio)propionate];
18:1 Succinyl PE 1,2-dioleoyl-sn-glycero-3-phosphoethanolamine-N-(succinyl);
16:0 Succinyl PE 1,2-dipalmitoyl-sn-glycero-3-phosphoethanolamine-N-(succinyl);
18:1 Glutaryl PE 1,2-dioleoyl-sn-glycero-3-phosphoethanolamine-N-(glutaryl);
16:0 Glutaryl PE 1,2-dipalmitoyl-sn-glycero-3-phosphoethanolamine-N-(glutaryl);
18:1 Dodecanyl PE 1,2-dioleoyl-sn-glycero-3-phosphoethanolamine-N-(dodecanoyl);
16:0 Dodecanoyl PE 1,2-dipalmitoyl-sn-glycero-3-phosphoethanolamine-N-(dodecanoyl);
16:0 Cyanur PE 1,2-dipalmitoyl-sn-glycero-3-phosphoethanolamine-N-(cyanur);
16:0 Folate Cap PE 1,2-dipalmitoyl-sn-glycero-3-phosphoethanolamine-N-(6-((folate)amino)hexanoyl);
16:0 PA-PEG3-mannose 1,2-dipalmitoyl-sn-glycero-3-phospho((ethyl-1′,2′,3′-triazole)triethyleneglycolmannose);
18:1 PE-Square 1,2-dioleoyl-sn-glycero-3-phosphoethanolamine-N-square;
18:0 PE-square 1,2-distearoyl-sn-glycero-3-phosphoethanolamine-N-square;
18:1 Ptd Ethylene Glycol 1,2-Dioleoyl-sn-Glycero-3-Phospho(Ethylene Glycol);
16:0 Ptd Ethylene Glycol 1,2-dipalmitoyl-sn-glycero-3-phospho(ethylene glycol),
or a pharmaceutically acceptable salt thereof. All of these lipids are available from commercial sources including Avanti Polar Lipids, Alabaster, Ala. USA.

“Fluorescence-inducing phospholipids” or “fluorescent phospholipids” are phospholipids which include a fluorescent moiety such as a fluorescein dye or a rhodamine dye which is included as a reporter. In some embodiments, the fluorescent moiety comprises two or more fluorescent dyes that can act cooperatively with one another, for example by fluorescence resonance energy transfer (“FRET”). The fluorescent moiety may be any fluorophore that is capable of producing a detectable fluorescence signal in an assay medium; the fluorescence signal can be “self-quenched” and capable of fluorescing in an aqueous medium. “Quench” refers to a reduction in the fluorescence intensity of a fluorescent group as measured at a specified wavelength, regardless of the mechanism by which the reduction is achieved. As specific examples, the quenching may be due to molecular collision, energy transfer such as FRET, a change in the fluorescence spectrum (color) of the fluorescent group or any other mechanism. The amount of the reduction is not critical and may vary over a broad range. The only requirement is that the reduction be measurable by the detection system being used. Thus, a fluorescence signal is “quenched” if its intensity at a specified wavelength is reduced by any measurable amount.

Examples of fluorophores include xanthenes such as fluoresceins, rhodamines and rhodols, cyanines, phtalocyanines, squairanines, bodipy dyes, pyrene, anthracene, naphthalene, acridine, stilbene, indole or benzindole, oxazole or benzoxazole, thiazole or benzothiazole, carbocyanine, carbostyryl, prophyrin, salicylate, anthranilate, azulene, perylene, pyridine, quinoline, borapolyazaindacene, xanthene, oxazine or benzoxazine, carbazine, phenalenone, coumarin, benzofuran, or benzphenalenone. Examples of rhodamine dyes include, but are not limited to, rhodamine B, 5-carboxyrhodamine, rhodamine X (ROX), 4,7-dichlororhodamine X (dROX), rhodamine 6G (R6G), 4,7-dichlororhodamine 6G, rhodamine 110 (R110), 4,7-dichlororhodamine 110 (dR110), tetramethyl rhodamine (TAMRA) and 4,7-dichlorotetramethylrhodamine (dTAMRA).

Examples of fluorescein dyes include, but are not limited to, 4,7-dichlorofluoresceins, 5-carboxyfluorescein (5-FAM) and 6-carboxyfluorescein (6-FAM).

The phospholipid component of the fluorescence-inducing phospholipids can comprise a 6 to 20 carbon saturated or unsaturated fatty acid chain and a head group. For example, the phospholipid component of the fluorescence-inducing phospholipids can include 1-Palmitoyl-2-Oleoyl-sn-Glycero-3-Phosphocholine (POPC), 1,2-Dimyristoyl-sn-Glycero-3-Phosphocholine (DMPC), 1,2-Dioleoyl Phosphatidylethanolamine (DOPE), 1,2-Dipalmitoyl-sn-Glycero-3-Phosphoethanolamine (DPPE), 1,2-Dioleoyl Phosphatidylcholine (DOPC), 1,2-Dioleoyl Phosphatidylserine (DOPS), labelled derivatives thereof, or a combination thereof.

Head groups include, but are not limited to, positively charged, negatively charged, zwitterionic, or uncharged head groups. Exemplary head groups include, but are not limited to, phosphatidyl choline, phosphatidyl ethanolamine, and phosphatidyl serine.

Optional covalent cross-linking of phospholipids is one mechanism that can be used to stabilize immunomicelles chemically and mechanically. Covalent cross-linking of the amphiphilic membrane components of the vesicle (non-naturally-occurring liposome) in general prevents the disruption of the membrane after head group hydrolysis. This approach may nevertheless be used if the covalent bond can be selectively cleaved or if it is unevenly distributed throughout the vesicle membrane. Thus, selective covalent cross-linking of the membrane may be permissible in a mosaic distribution, where parts of the vesicle membrane which will disrupt are not crosslinked. The non-cross-linked portions may leach or disintegrate leaving a porous membrane through which said pores the content may be leached out.

Vesicles may be cross-linked either through the polar head groups or through reactive groups within the hydrophobic membranes. For example, head groups with vinyl substituents can be polymerized on the outer surface with water-soluble initiators or on both surfaces with UV radiation. Reactive groups within the hydrophobic layer of the membrane are inaccessible to water-soluble reagents. In general, visible and UV radiation has been used to polymerize amphiphiles through diene or diyne groups. Cross linking may also occur via condensation reactions widely known in the state of art. For example, if the monomer amphiphilic compounds contain epoxy groups, they may be self-condensed using appropriate catalysts (e.g., tertiary amines containing hydroxyl or phenol groups), or through reaction with polyfunctional amines.

“Cross-linking phospholipids” include, but are not limited to 1,2-Dioleoyl-sn-Glycero-3-Phosphocholine (DOPC), 1,2-Dilauroyl-sn-Glycero-3-Phosphocholine (DLPC), 1-Palmitoyl-2-10,12 Tricosadiynoyl-sn-Glycero-3-Phosphocholine (16:0-23:2 DIYNE PC), and 1,2-bis(10,12-tricosadiynoyl)-sn-glycero-3-phosphoethanol amine (Diyne-PE). Phospholipids with various head groups such as phosphatidylethanol, phosphatidylpropanol and phosphatidylbutanol, phosphatidylethanol amine-N-monomethyl, 1,2-disteraoyl(dibromo)-sn-glycero-3-phosphocoline, and phospholipids with partially or fully fluorinated fatty acid chains, as well as the phospholipids listed above which are conjugative phospholipids, can also be used.

“Cross-linking agents” are compounds with two or more functional groups, preferably two functional groups which are capable of linking one component of the immunomicelles (e.g. an antibody, a binding protein, a reporter molecule, a penetration peptide, etc.) which are otherwise described herein to another component such as a functionalized or conjugatable phospholipid which encapsulates the paramagnetic core of the immunomicelles. Crosslinking agents may have functional groups which are both nucleophilic functional groups, both electrophilic groups or nucleophilic/electrophilic groups depending on the component to be conjugated to another component (often a conjugatable phospholipid) of the immunomicelle. The term “protein crosslinking” refers to utilizing protein crosslinkers to conjugate peptides or proteins together. Crosslinking agents for use herein possess reactive moieties specific to various electrophilic or nucleophilic functional groups (e.g., sulfhydryls, amines, carbohydrates, carboxyl groups, hydroxyl groups, carbonyls, etc.) on proteins, peptides, or other molecular complexes or molecules such as antibodies, reporters, penetration agents and the like as described herein. The atoms separating a crosslinker agent's reactive groups, and eventually the conjugated components form the “spacer arm”. A zero-length crosslinker refers to protein crosslinkers that join two molecules without adding additional spacer arm atoms. Homobifunctional crosslinker reagents have the same reactive group on both ends of the spacer arm (i.e., amine reactive-amine reactive); while heterobifunctional crosslinkers have different reactive groups on each end of a spacer arm (i.e., sulfhydryl or hydroxyl reactive-amine Reactive). It is noted that in addition to the following crosslinking agents, additional short-chain crosslinking agents such as short-chain alkyl amides (CH₂)ᵢC(O)NH₂, (CH₂)ᵢC(O), C(O)(CH₂)ᵢC(O), NHC(O)(CH₂)ᵢC(O) or NHC(O)(CH₂)ᵢC(O)NH groups where i is from 1 to 4, can be used to link a component to the crosslinker. The following crosslinking agents are exemplary for use in the present invention and are used depending on the functional groups which are present on the component to be cross-linked or conjugated. Exemplary cross-linking or conjugating agents include the following:
ANB-NOS (N-5-Azido-2-nitrobenzoyloxysuccinimide)
BMPS N-(β-Maleimidopropyloxy)succinimide ester
EMCS (N[e-Maleimidocaproyloxy]succinimide ester)
GMBS (N-[Gamma-Maleimidobutyryloxy] Succinimide)
LC-SPDP Succinimidyl 6-(3-[2-pyridyldithio]-propionamido)hexanoate)
MBS (m-Maleimidobenzoyl-N-hydroxysuccinimide ester)
PDPH (3-[2-Pyridyldithio]propionyl hydrazide)
SBA (N-Succinimidyl bromoacetate)
SIA (N-Succinimidyl iodoacetate)
Sulfa-SIA N-Sulfosuccinimidyl iodoacetate)
SMCC (Succinimidyl-4-[N-maleimidomethyl]cyclohexane-1-carboxylate)
SMPB (N-Succinimidyl 4-[4-maleimidophenyl]butyrate)
SMPH (Succinimidyl-6-[β-maleimidopropionamido]hexanoate)
SPDP (N-Succinimidyl 3-[2-pyridyldithio]-propionate)
Sulfo-LC-SPDP Sulfosuccinimidyl 6-(3′-[2-pyridyldithio]-propionamido)hexanoate
Sulfo-MBS (m-Maleimidobenzoyl-N-hydroxysulfosuccinimide ester)
Sulfo-SANPAH (N-Sulfosuccinimidyl-6-[4′-azido-2′-nitrophenylamino] hexanoate)
sulfo-SMCC (Sulfosuccinimidyl4-[N-maleimidomethyl]cyclohexane-1-carboxylate)
BS2G (Bis[Sulfosuccinimidyl] glutarate)
BS3 (Bis[sulfosuccinimidyl] suberate)
DSG (Disuccinimidyl glutarate)
DSP (Dithiobis[succinimidyl propionate])
DSS (Disuccinimidyl suberate)
DSSeb (Disuccinimidyl sebacate)
DST (Disuccinimidyl tartrate)
DTSSP (3,3′-Dithiobis[sulfosuccinimidylpropionate])
EGS (Ethylene glycolbis(succinimidylsuccinate)
Sulfa-EGS Ethylene glycolbis(sulfosuccinimidylsuccinate)
CDI (N,N′-Carbonyldiimidazole)
DCC (N,N-dicyclohexylcarbodiimide)
EDC-HCl 1-Ethyl-3-[3-dimethylaminopropyl]carbodiimide hydrochloride)
NHS (N-hydroxysuccinimide) and
Sulfo-NHS (N-hydroxysulfosuccinimide).

Preferred crosslinkers for use in the present invention are heterobifunctional agents such as N-hydroxysulfosuccinimide (Sulfo-NHS) and 1-Ethyl-3-[3-dimethylaminopropyl]carbodiimide hydrochloride (EDC). Other exemplary crosslinking agents which are often used include:
SIA (succinimidyl iodoacetate)
SBAP (succinimidyl 3-(bromoacetamido)propionate)
SIAB (succinimidyl (4-iodoacetyl)aminobenzoate)
Sulfo-SIAB (sulfosuccinimidyl (4-iodoacetyl)aminobenzoate)
AMAS (N-α-maleimidoacet-oxysuccinimide ester)
BMPS (N-β-maleimidopropyl-oxysuccinimide ester)
GMBS (N-γ-maleimidobutyryl-oxysuccinimide ester)
MBS (m-maleimidobenzoyl-N-hydroxysuccinimide ester)
SMCC (succinimidyl 4-(N-maleimidomethyl)cyclohexane-1-carboxylate)
EMCS (N-ε-malemidocaproyl-oxysuccinimide ester)
Sulfo-GMBS (N-γ-maleimidobutyryl-oxysulfosuccinimide ester)
Sulfo-MBS (m-maleimidobenzoyl-N-hydroxysulfosuccinimide ester)
Sulfo-SMCC (sulfosuccinimidyl 4-(N-maleimidomethyl)cyclohexane-1-carboxylate)
Sulfo-EMCS (N-ε-maleimidocaproyl-oxysulfosuccinimide ester)
Sulfo-SMPB (sulfosuccinimidyl 4-(N-maleimidophenyl)butyrate)
SMPB (succinimidyl 4-(p-maleimidophenyl)butyrate)
SMITH (Succinimidyl 6-((beta-maleimidopropionamido)hexanoate))
LC-SMCC (succinimidyl 4-(N-maleimidomethyl)cyclohexane-1-carboxy-(6-amidocaproate)) and
Sulfo-KMUS (N-κ-maleimidoundecanoyl-oxysulfosuccinimide ester).

“Stealth” immunomicelles circulate over long times without immune or renal clearing. Stealth liposomes that are sterically stabilized with lipid derivatives of polyethylene glycol (PEG) are illustrated, e.g., in Lopes de Menezes, et al., Journal of Liposome Research, 1999, Vol. 9, No. 2, Pages 199-228 and Immordino, et al., Int. J. Nanomedicine, 2006; 1(3). As with stealth liposomes, stealth immunomicelles are useful anti-cancer formulations because of their passive targeting effect, which may lead to preferential accumulation in tumor tissue. Stealth immunomicelles can lodge in the interstitial spaces between cancer cells. Once in the tumor area, the immunomicelles locate in the extracellular fluid surrounding the cell without actually entering the cell. The anti-cancer agent is first released. from the immunomicelle into the tumor extracellular fluid, and then diffuses into the cell.

Antibodies to Iba-1, GFAP and other proteins associated with microglial cells or in alternative embodiments cells and/or tissue associated with eurodegenerative disease states and/or conditions as described herein and are conjugated to immunomicelle particles include, e.g., antibodies to markers whose expression is specifically altered in microglial activation (e.g. up regulated when activated or down regulated after an activation period). Such antibodies include anti-Iba-1 and anti-GFAP antibodies for stroke, as well as anti-tau/anti-β amyloid antibodies for Alzheimer's, anti-huntingtin protein antibodies for Huntington's disease, anti-Iba-1 antibodies for Parkinson's disease and anti-alpha-synuclein for ALS.

Nonlimiting examples of blood-brain barrier-penetration ligands, which can encompass proteins and amino acid constructs, include insulin, antibodies against the human insulin receptor, and the following peptides SEQ ID NOS: 1-13:
Thr-Phe-Phe-Tyr-Gly-Gly-Cys-Arg-Gly-Lys-Arg-Asn-Asn-Phe-Lys-Thr-Glu-Glu-Tyr (Angiopep-1) SEQ ID NO:1,
Thr-Phe-Phe-Tyr-Gly-Gly-Ser-Arg-Gly-Lys-Arg-Asn-Asn-Phe-Lys-Thr-Glu-Glu-Tyr (Angiopep-2) SEQ ID NO:2,
Thr-Phe-Phe-Tyr-Gly-Gly-Ser-Arg-Gly-Lys-Arg-Asn-Asn-Phe-Arg-Thr-Gtu-Glu-Tyr (Angiopep-5) SEQ ID NO:3,
Thr-Phe-Phe-Tyr-Gly-Gly-Ser-Arg-Gly-Arg-Arg-Asn-Asn-Phe-Arg-Thr-Glu-Glu-Tyr (Angiopep-7) SEQ ID NO:4,
Thr-Phe-Phe-Tyr-Gly-Gly-Cys-Arg-Ala-Lys-Arg-Asn-Asn-Phe-Lys-Arg-Ala-Lys-Tyr SEQ ID NO:5,
Thr-Phe-Phe-Tyr-Gly-Gly-Cys-Arg-Gly-Lys-Lys-Asn-Asn-Phe-Lys-Ala-Lys-Tyr SEQ ID NO:6,
Pro-Phe-Phe-Tyr-Gly-Gly-Cys Arg-Glyn-Lys-Arg-Asn-Asn-Phe-Lys-Thr-Glu-Glu-Tyr SEQ ID NO:7,
Thr-Phe-Phe-Tyr-Cily-Gly-Cys-Arg-Gly-Lys-Arg-Asn-Asn-Phe-Lys-Thr-Lys-Glu-Tyr, Thr-Phe-Phe-Tyr-Gly-Gly-Lys-Arg-Gly-Lys-Arg-Asn-Asn-Phe-Lys-Thr-Lys-Glu-Tyr SEQ ID NO:8,
Thr-Phe-Phe-Tyr-Gly-Gly-Cys-Arg-Gly-Lys-Arg-Asn-Asn-Phe-Lys-Thr-Lys-Arg-Tyr SEQ ID NO:9,
Thr-Phe-Phe-Tyr-Gly-Gly-Lys-Arg-Gly-Lys-Arg-Asn-Asn-Phe-Lys-Thr-Ala-Glu-Tyr SEQ ID NO:10,
Thr-Phe-Phe-Tyr-Gly-Gly-Lys-Arg-Gly-Lys-Arg-Asn-Asn-Phe-Lys-Arg-Glu-Lys-Tyr SEQ ID NO:11,
Arg-Phe-Lys-Tyr-Gly-Gly-Cys-Leu-Gly-Asn-Lys-Asn-Asn-Phe-Leu-Arg-Leu-Lys-Tyr SEQ ID NO:12, and
Arg-Phe-Lys-Tyr-Gly-Gly-Cys-Leu-Gly-Asn-Lys-Asn-Asn-Tyr-Leu-Arg-Leu Lys Tyr SEQ ID NO:13. A preferred component for penetrating the blood brain barrier is polysorbate 80.

As used herein, “antibody” includes, but is not limited to, monoclonal antibodies and polyclonal antibodies raised against Iba-1 or GFAP protein or other proteins or polypeptides which are important proteins in other neurodegenerative disorders such as tau/β-amyloid (Alzheimer's disease), huntingtin protein (Huntington's disease), Iba-1 (Parkinson's disease) and alpha-synuclein (α-synuclein) (ALS), among others. The following disclosure from U.S. Patent Application Document No. 20100284921, the entire contents of which are hereby incorporated by reference, exemplifies techniques that are useful in making antibodies employed in formulations of the instant invention.

As described in U.S. Patent Application Document No. 20100284921, “antibodies . . . may be polyclonal or monoclonal. Monoclonal antibodies are preferred. The antibody is preferably a chimeric antibody. For human use, the antibody is preferably a humanized chimeric antibody.

[A]n anti-target-structure antibody . . . may be monovalent, divalent or polyvalent in order to achieve target structure binding. Monovalent immunoglobulins are dimers (HL) formed of a hybrid heavy chain associated through disulfide bridges with a hybrid light chain. Divalent immunoglobulins are tetramers (H2L2) formed of two dimers associated through at least one disulfide bridge.

The invention also includes [use of] functional equivalents of the antibodies described herein. Functional equivalents have binding characteristics comparable to those of the antibodies, and include, for example, hybridized and single chain antibodies, as well as fragments thereof. Methods of producing such functional equivalents are disclosed in PCT Application Nos. WO 1993/21319 and WO 1989/09622. Functional equivalents include polypeptides with amino acid sequences substantially the same as the amino acid sequence of the variable or hyper variable regions of the antibodies raised against target integrins according to the practice of the present invention.

Functional equivalents of the anti-target-structure antibodies further include peptides, aptamers consisting of fragments either DNA or RNA, or of antibodies that have the same, or substantially the same, binding characteristics to those of the whole antibody. Such fragments may contain one or both Fab fragments or the F(ab′)₂ fragment. Preferably the antibody fragments contain all six complement determining regions of the whole antibody, although fragments containing fewer than all of such regions, such as three, four or five complement determining regions, are also functional. The functional equivalents are members of the IgG immunoglobulin class and subclasses thereof, but may be or may combine any one of the following immunoglobulin classes: IgM, IgA, IgD, or IgE, and subclasses thereof. Heavy chains of various subclasses, such as the IgG subclasses, are responsible for different effector functions and thus, by choosing the desired heavy chain constant region, hybrid antibodies with desired effector function are produced. Preferred constant regions are gamma 1 (IgG1), gamma 2 (IgG2 and IgG), gamma 3 (IgG3) and gamma 4 (IgG4). The light chain constant region can be of the kappa or lambda type.

The monoclonal antibodies may be advantageously cleaved by proteolytic enzymes to generate fragments retaining the target structure binding site. For example, proteolytic treatment of IgG antibodies with papain at neutral pH generates two identical so-called “Fab” fragments, each containing one intact light chain disulfide-bonded to a fragment of the heavy chain (Fc). Each Fab fragment contains one antigen-combining site. The remaining portion of the IgG molecule is a dimer known as “Fc”. Similarly, pepsin cleavage at pH 4 results in the so-called F(ab′)₂ fragment.

Single chain antibodies or Fv fragments are polypeptides that consist of the variable region of the heavy chain of the antibody linked to the variable region of the light chain, with or without an interconnecting linker. Thus, the Fv comprises an antibody combining site.

Hybrid antibodies may be employed. Hybrid antibodies have constant regions derived substantially or exclusively from human antibody constant regions and variable regions derived substantially or exclusively from the sequence of the variable region of a monoclonal antibody from each stable hybridoma.

Methods for preparation of fragments of antibodies are known to those skilled in the art. See, Goding, “Monoclonal Antibodies Principles and Practice”, Academic Press (1983), p. 119-123. Fragments of the monoclonal antibodies containing the antigen binding site, such as Fab and F(ab′)₂ fragments, may be preferred in therapeutic applications, owing to their reduced immunogenicity. Such fragments are less immunogenic than the intact antibody, which contains the immunogenic Fc portion. Hence, as used herein, the term “antibody” includes intact antibody molecules and fragments thereof that retain antigen binding ability.

When the antibody used in the practice of the invention is a polyclonal antibody (IgG), the antibody is generated by inoculating a suitable animal with a target structure or a fragment thereof. Antibodies produced in the inoculated animal that specifically bind the target structure are then isolated from fluid obtained from the animal. Anti-target-structure antibodies may be generated in this manner in several non-human mammals such as, but not limited to, goat, sheep, horse, rabbit, and donkey. Methods for generating polyclonal antibodies are well known in the art and are described, for example in Harlow et al. (In: Antibodies, A Laboratory Manual, 1988, Cold Spring Harbor, N.Y.),

When the antibody used in the methods used in the practice of the invention is a monoclonal antibody, the antibody is generated using any well-known monoclonal antibody preparation procedures such as those described, for example, in Harlow et al. (supra) and in Tuszynski et al. (Blood 1988, 72:109-115). Generally, monoclonal antibodies directed against a desired antigen are generated from mice immunized with the antigen using standard procedures as referenced herein. Monoclonal antibodies directed against full length or fragments of target structure may be prepared using the techniques described in Harlow et al. (supra).

The effects of sensitization in the therapeutic use of animal-origin monoclonal antibodies in the treatment of human disease may be diminished by employing a hybrid molecule generated from the same Fab fragment, but a different Fc fragment, than contained in monoclonal antibodies previously administered to the same subject. It is contemplated that such hybrid molecules formed from the anti-target-structure monoclonal antibodies may be used in the present invention. The effects of sensitization are further diminished by preparing animal/human chimeric antibodies, e.g., mouse/human chimeric antibodies, or humanized (i.e. CDR-grafted) antibodies. Such monoclonal antibodies comprise a variable region, i.e., antigen binding region, and a constant region derived from different species. By ‘chimeric’ antibody is meant an antibody that comprises elements partly derived from one species and partly derived from at least one other species, e.g., a mouse/human chimeric antibody.

Chimeric animal-human monoclonal antibodies may be prepared by conventional recombinant DNA and gene transfection techniques well known in the art. The variable region genes of a mouse antibody-producing myeloma cell line of known antigen-binding specificity are joined with human immunoglobulin constant region genes. When such gene constructs are transfected into mouse myeloma cells, the antibodies produced are largely human but contain antigen-binding specificities generated in mice. As demonstrated by Morrison et al., 1984, Proc. Natl. Acad. Sci. USA 81:6851-6855, both chimeric heavy chain V region exon (VH)-human heavy chain C region genes and chimeric mouse light chain V region exon (VK)-human K light chain gene constructs may be expressed when transfected into mouse myeloma cell lines. When both chimeric heavy and light chain genes are transfected into the same myeloma cell, an intact H2L2 chimeric antibody is produced. The methodology for producing such chimeric antibodies by combining genomic clones of V and C region genes is described in the above-mentioned paper of Morrison et al., and by Boulianne et al. (Nature 1984, 312:642-646). Also see Tan et al. (J. Immunol. 1985, 135:3564-3567) for a description of high level expression from a human heavy chain promotor of a human-mouse chimeric K chain after transfection of mouse myeloma cells. As an alternative to combining genomic DNA, cDNA clones of the relevant V and C regions may be combined for production of chimeric antibodies, as described by Whitte et al. (Protein Eng. 1987, 1:499-505) and Liu et al. (Proc. Natl. Acad. Sci. USA 1987, 84:3439-3443). For examples of the preparation of chimeric antibodies, see the following U.S. Pat. Nos 5,292,867; 5,091,313; 5,204,244; 5,202,238; and 5,169,939. The entire disclosures of these patents, and the publications mentioned in the preceding paragraph, are incorporated herein by reference. Any of these recombinant techniques are available for production of rodent/human chimeric monoclonal antibodies against target structures.

To further reduce the immunogenicity of murine antibodies, “humanized” antibodies have been constructed in which only the minimum necessary parts of the mouse antibody, the complementarity-determining regions (CDRs), are combined with human V region frameworks and human C regions (Jones et al., 1986, Nature 321:522-525; Verhoeyen et al., 1988, Science 239:1534-1536; Hale et al., 1988, Lancet 2:1394-1399; Queen et al., 1989, Proc. Natl. Acad. Sci. USA 86:10029-10033). The entire disclosures of the aforementioned papers are incorporated herein by reference. This technique results in the reduction of the xenogeneic elements in the humanized antibody to a minimum. Rodent antigen binding sites are built directly into human antibodies by transplanting only the antigen binding site, rather than the entire variable domain, from a rodent antibody. This technique is available for production of chimeric rodent/human anti-target structure antibodies of reduced human immunogenicity.”

Further, standard techniques for growing cells, separating cells, and where relevant, cloning, DNA isolation, amplification and purification, for enzymatic reactions involving DNA ligase, DNA polymerase, restriction endonucleases and the like, and various separation techniques are those known and commonly employed by those skilled in the art. A number of standard techniques are described in Sambrook et al., 1989 Molecular Cloning, Second Edition, Cold Spring Harbor Laboratory, Plainview, New York; Maniatis et al., 1982 Molecular Cloning, Cold Spring Harbor Laboratory, Plainview, New York; Wu (Ed.) 1993 Meth. Enzymol. 218, Part I; Wu (Ed.) 1979 Meth. Enzymol. 68; Wu et al., (Eds.) 1983 Meth. Enzymol. 100 and 101; Grossman and Moldave (Eds.) 1980 Meth. Enzymol. 65; Miller (ed.) 1972 Experiments in Molecular Genetics, Cold Spring Harbor Laboratory, Cold Spring Harbor, New York; Old and Primrose, 1981 Principles of Gene Manipulation, University of California Press, Berkeley; Schleif and Wensink, 1982 Practical Methods in Molecular Biology; Glover (Ed.) 1985 DNA Cloning Vol. I and II, IRL Press, Oxford, UK; Hames and Higgins (Eds.) 1985 Nucleic Acid Hybridization, IRL Press, Oxford, UK; and Setlow and Hollaender 1979 Genetic Engineering: Principles and Methods, Vols. 1-4, Plenum Press, New York. Abbreviations and nomenclature, where employed, are deemed standard in the field and commonly used in professional journals such as those cited herein.

Formulations of the invention, in addition to being used exclusively for diagnosing stroke or monitoring therapy of stroke can also be designed for delivering agents for the treatment of disorders associated with the CNS, including stroke.

“Immunomicelles” and “micelles” are aggregates formed by amphipathic molecules in water or an aqueous solvent such that their polar ends or portions are in contact with the water or aqueous solvent and their nonpolar ends or portions are in the interior of the aggregate. A micelle can take any shape or form, including but not limited to, a non-lamellar “detergent-like” aggregate that does not enclose a portion of the water or aqueous solvent, or a unilamellar or multilamellar “vesicle-like” aggregate that encloses a portion of the water or aqueous solvent, such as, for example, a liposome. Specifically included within the definition of “micelle” are small unilamellar vesicles or liposomes (“SUVs”), small multilamellar vesicles or liposomes (“SMVs”), large unilamellar vesicles or liposomes (“LUVs”) and large multilamellar vesicles or liposomes (“LMVs”).

The term “patient” or “subject” is used throughout the specification to describe an animal, preferably a human, to whom diagnosis or treatment, including prophylactic treatment, with the compositions according to the present invention is provided (a patient or subject in need). For treatment of those infections, conditions or disease states which are specific for a specific animal such as a human patient, the term patient refers to that specific animal. In many instances, diagnostic methods are applied to patients or subjects who are suspected of ischemia and the diagnostic method is used to assess the severity of the disease state or disorder.

The term “compound” is used herein to refer to any specific chemical compound disclosed herein. Within its use in context, the term generally refers to a single small molecule as disclosed herein, but in certain instances may also refer to stereoisomers and/or optical isomers (including racemic mixtures) of disclosed compounds. The term compound includes active metabolites of compounds and/or pharmaceutically active salts thereof.

The term “effective amount” is used throughout the specification to describe concentrations or amounts of formulations or other components which are used in amounts, within the context of their use, to produce an intended effect according to the present invention. The formulations or component may be used to produce a favorable change in a disease or condition treated, whether that change is a remission, a favorable physiological result, a reversal or attenuation of a disease state or condition treated, the prevention or the reduction in the likelihood of a condition or disease-state occurring, depending upon the disease or condition treated. Where formulations are used in combination, each of the formulations is used in an effective amount, wherein an effective amount may include a synergistic amount. The amount of formulation used in the present invention may vary according to the nature of the formulation, the age and weight of the patient and numerous other factors which may influence the bioavailability and pharmacokinetics of the formulation, the amount of formulation which is administered to a patient generally ranges from about 0.001 mg/kg to about 50 mg/kg or more, about 0.5 mg/kg to about 25 mg/kg, about 0.1 to about 15 mg/kg, about 1 mg to about 10 mg/kg per day and otherwise described herein. The person of ordinary skill may easily recognize variations in dosage schedules or amounts to be made during the course of therapy.

The term “prophylactic” is used to describe the use of a formulation described herein which reduces the likelihood of an occurrence of a condition or disease state in a patient or subject. The term “reducing the likelihood” refers to the fact that in a given population of patients, the present invention may be used to reduce the likelihood of an occurrence, recurrence or metastasis of disease in one or more patients within that population of all patients, rather than prevent, in all patients, the occurrence, recurrence or metastasis of a disease state.

The term “pharmaceutically acceptable” refers to a salt form or other derivative (such as an active metabolite or prodrug form) of the present compounds or a carrier, additive or excipient which is not unacceptably toxic to the subject to which it is administered.

The term “ischemia” is used throughout the specification to refer to the pathological process that results in the limited blood flow to a body part, in the case of stroke the brain, resulting in stroke.

The term “control” or “standard” is used throughout the specification to refer to an image, data or other reference information which may be used to compare the existence and/or extent of ischemia in a patient for purposes of diagnosing stroke/ischemic, when the stroke occurred, the extent of neuroinflammation associated with the stroke/ischemia, the timing and extent of healing of tissue and the response of ischemic tissue to therapy (monitoring of therapy). The control or standard may be an MRI image, a read-out of an MRI or other data such as change of control or other data obtained from the MRI, especially including iron uptake as described in more detail herein. Iron uptake may be readily used to compare the MRI of a subject or patient with either a normal/healthy patient (where iron uptake is essentially zero) or a patient with disease in varying states, as applicable. The control or standard is compared to the MRI image and data obtained from the patient to be diagnosed and/or treated in order to more fully assess when a stroke occurred, the extent of neuroinflammation and the extent and timing of the healing of tissue associated with a stroke.

Pursuant to the present invention, one particularly effective method of diagnosing and assessing stroke and in particular, neuroinflammation associated with stroke using MRI is through determination of the local concentration of activated microglia within infarcted regions of the brain post-stroke. The neuroinflammation associated with stroke is mediated by the activation of microglia, the brain's resident immune cells. In a principal embodiment, the present invention reveals this process through the specific uptake of FePt nanoparticles targeted to these activated brain cells which is evidenced by the MRI. The amount of iron taken up is proportional to the activated cell density. By using the method provided in Sillerud, Int J Nanomedicine 2016; 11:357-372, for measuring the amount of iron present in brain tissue, a skilled practitioner can estimate the density of activated microglial cells, and hence the degree of neuroinflammation in the tissue. Each activated microglial cell takes up approximately 14-30 femtograms of iron. By measuring the tissue containing iron concentration added from the nanoparticles, the skilled practitioner can therefore estimate the number of activated microglia per cubic centimeter and compare these values with a standard taken from normal patients and stroke patients to determine the level of neuroinflammation. A normal patient measures zero iron and stroke patients at various stages of neuroinflammation contain varying levels of iron content. By determining the iron content of microglial cells in a stroke patient and then comparing that content with one or more standards from different patients with different levels of stroke, from this method a skilled practitioner may readily determine the extent of the stroke and how far along the path to recovery the patient is. This method can be used to both diagnose stroke and monitoring therapy or favorable prognosis of the patient to recovery after stroke.

The term “longitudinal monitoring” is used to describe a study where the same data are collected in tissue at different points in time. In the present invention, MIR longitudinal monitoring is an MRI study where the same MRI data are collected more than once in the same tissue, at different points in time. The purpose of longitudinal monitoring is to assess not just what the data reveal at a fixed point in time, but to understand how (and why) things change in the tissue being monitored over time.

Formulations of the invention may include a pharmaceutically acceptable diluent, carrier, solubilizer, emulsifier, preservative and/or adjuvant. Acceptable formulation materials preferably are nontoxic to recipients at the dosages and concentrations employed. The pharmaceutical formulations may contain materials for modifying, maintaining or preserving, for example, the pH, osmolarity, viscosity, clarity, color, isotonicity, odor, sterility, stability, rate of dissolution or release, adsorption or penetration of the composition. Suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides, disaccharides, and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, polyethylene glycol (PEG), sorbitan esters, polysorbates such as polysorbate 20 and polysorbate 80, Triton, trimethamine, lecithin, cholesterol, or tyloxapal); stability enhancing agents (such as sucrose or sorbitol); tonicity enhancing agents (such as alkali metal halides, preferably sodium or potassium chloride, mannitol, or sorbitol); delivery vehicles; diluents; excipients and/or pharmaceutical adjuvants. See, for example, REMINGTON'S PHARMACEUTICAL SCIENCES, 18.sup.th Edition, (A. R. Gennaro, ed.), 1990, Mack Publishing Company.

Optimal pharmaceutical formulations can be determined by one skilled in the art depending upon, for example, the intended route of administration, delivery format and desired dosage. See, for example, REMINGTON'S PHARMACEUTICAL SCIENCES, Id. Such formulations may influence the physical state, stability, rate of in vivo release and rate of in vivo clearance of the antibodies of the invention.

Primary vehicles or carriers in a pharmaceutical formulation can include, but are not limited to, water for injection, physiological saline solution or artificial cerebrospinal fluid, possibly supplemented with other materials common in compositions for parenteral administration. Neutral buttered saline or saline mixed with serum albumin are further exemplary vehicles. Pharmaceutical formulations can comprise Tris buffer of about pH 7.0-8.5, or acetate buffer of about pH 4.0-5.5, which may further include sorbitol or a suitable substitute. Pharmaceutical formulations of the invention may be prepared for storage by mixing the selected composition having the desired degree of purity with optional formulation agents (REMINGTON'S PHARMACEUTICAL SCIENCES, Id.) in the form of a lyophilized cake or an aqueous solution. Further, the formulations may be formulated as a lyophilizate using appropriate excipients such as sucrose.

Formulation components are present in concentrations that are acceptable to the site of administration. Buffers are advantageously used to maintain the composition at physiological pH or at a slightly lower pH, typically within a pH range of from about 5 to about 8.

The pharmaceutical formulations of the invention can be delivered parenterally. When parenteral administration is contemplated, the therapeutic formulations for use in this invention may be in the form of a pyrogen-free, parenterally acceptable aqueous solution. Preparation involves the formulation of the desired immunomicelle, which may provide controlled or sustained release of the product which may then be delivered via a depot injection. Formulation with hyaluronic acid has the effect of promoting sustained duration in the circulation.

Formulations may be formulated for inhalation. In these embodiments, a stealth immunomicelle formulation is formulated as a dry powder for inhalation, or inhalation solutions may also be formulated with a propellant for aerosol delivery, such as by nebulization. Pulmonary administration is further described in PCT Application No. PCT/US94/001875, which describes pulmonary delivery of chemically modified proteins and is incorporated by reference.

Formulations of the invention can be delivered through the digestive tract, such as orally. The preparation of such pharmaceutically acceptable compositions is within the skill of the art. Formulations disclosed herein that are administered in this fashion may be formulated with or without those carriers customarily used in the compounding of solid dosage forms such as tablets and capsules. A capsule may be designed to release the active portion of the formulation at the point in the gastrointestinal tract when bioavailability is maximized and pre-systemic degradation is minimized Additional agents can be included to facilitate absorption. Diluents, flavorings, low melting point waxes, vegetable oils, lubricants, suspending agents, tablet disintegrating agents, and binders may also be employed.

A formulation may involve an effective quantity of a stealth immunomicelle formulation as disclosed herein in a mixture with non-toxic excipients that are suitable for the manufacture of tablets. By dissolving the tablets in sterile water, or another appropriate vehicle, solutions may be prepared in unit-dose form. Suitable excipients include, but are not limited to, inert diluents, such as calcium carbonate, sodium carbonate or bicarbonate, lactose, or calcium phosphate; or binding agents, such as starch, gelatin, or acacia; or lubricating agents such as magnesium stearate, stearic acid, or talc.

The pharmaceutical composition to be used for in vivo administration typically is sterile. In certain embodiments, this may be accomplished by filtration through sterile filtration membranes. In certain embodiments, where the composition is lyophilized, sterilization using his method may be conducted either prior to or following lyophilization and reconstitution. In certain embodiments, the composition for parenteral administration may be stored in lyophilized form or in a solution. In certain embodiments, parenteral compositions generally are placed into a container having a sterile access port, for example, an intravenous solution bag or vial having a stopper pierceable by a hypodermic injection needle.

Once the formulation of the invention has been formulated, it may be stored in sterile vials as a solution, suspension, gel, emulsion, solid, or as a dehydrated or lyophilized powder. Such formulations may be stored either in a ready-to-use form or in a form (e.g., lyophilized) that is reconstituted prior to administration.

Administration routes for formulations of the invention include orally, through injection by intravenous, intraperitoneal, intracerebral (intra-parenchymal), intracerebroventricular, intramuscular, intra-ocular, intraarterial, intraportal, or intralesional routes; by sustained release systems or by implantation devices. The pharmaceutical formulations may be administered by bolus injection or continuously by infusion, or by implantation device. The pharmaceutical formulations also can be administered locally via implantation of a membrane, sponge or another appropriate material onto which the desired molecule has been absorbed or encapsulated. Where an implantation device is used, the device may be implanted into any suitable tissue or organ, and delivery of the desired molecule may be via diffusion, timed-release bolus, or continuous administration.

In the present invention immunomicelle compositions as described herein are typically injected into a patient who is known to have had a stroke or neurodegenerative disease state and after a period sufficient to allow distribution of the immunomicelles into central nervous system tissue, especially brain tissue may be scanned. Once the immunomicelles are distributed MRI or CT is conducted on the patient and an image of the brain tissue is obtained. Analysis of the MRI image and related data are compared to a standard (which may be data obtained from a normal patient in identical tissue or from one or more patients who suffered a stroke or a neurodegenerative disease) to determine the existence, extent and development of activated microglia in the brain tissue to establish the existence of neuroinflammation or the stage of recovery of the stroke tissue. The number of and/or extent of activated microglia in brain tissue of a patient is best determined by measuring the amount of iron taken up by activated microglia pursuant to the method of Sillerud, 2016, supra, which is incorporated by reference herein, and comparing iron uptake with a standard or control. In methods pursuant to the present invention which are used to monitor therapy, prior to administering therapy, brain tissue from the patient is first analyzed by MRI or CT and at varying intervals after therapy is commenced (which may be one time up 10-15 times or more, often 1-3 times) to determine whether therapy is to be terminated (because a favorable outcome has been reached or no further change is expected), continued or modified.

These and other aspects of the invention are described further in the non-limiting examples which are presented herein below.

### EXAMPLES

### Methods and Materials

A detailed description of the methods is found in the Supplemental Methods.

Synthesis of FePt cores. Superparamagnetic iron platinum nanoparticles (SIPPs) were synthesized according to our previously published methods^{16, 17, 23, 24}.

Incorporation of the FePt cores into SIPP micelles. The construction of SIPP micelles (SM80s, SIPP micelles with polysorbate 80) was based on a thin-film hydration process followed by extrusion through a controlled pore membrane. The composition of the SM80s is given in Supplementary Table 1. We used a polyethyleneglycol coating to evade the reticuloendothial system and to avoid the buildup of a protein corona⁸. This procedure made ˜4.6×10¹⁴ SIPP micelles containing ˜6×10⁴ dipalmitoyl-sn-glycerolphosphorylcholine molecules/micelle with ˜98 biotin sites/micelle.

Protein conjugation to the SIPP micelles. Proteins (anti-Iba-1 and ApoE2) were streptavidinated using Lightning-Link Streptavidin conjugation kits from Innova Biosciences according to the manufacturer's protocol (https://www.novusbio.com/lightning-link-conjugation-kits). The modified proteins were then combined and added to the biotinylated micelles to produce the finished SM80s at an iron concentration of 12.5 mg/mL. These ˜50 nm diameter nanoparticles were stored at 4° C. until needed²⁴.

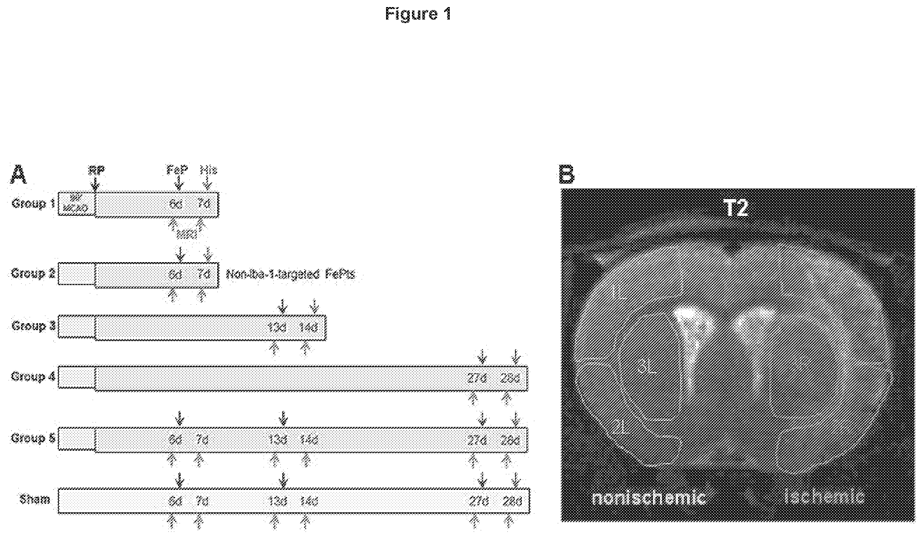

Rat stroke model of MGM with reperfusion and timeline for injections. The study was approved by the University of New Mexico Animal Care Committee and conforms to the National Institutes of Health guidelines for use of animals in research. Every effort was made to minimize the number of experimental animals used and their suffering. Reporting of this work complies with ARRIVE guidelines²⁵. Male spontaneously hypertensive rats (SHR; 12 weeks old) were subjected to 90 min MCAO followed by reperfusion (RP) for 1, 2 and 4 weeks as previously described¹⁴. 100 μl of the anti-Iba-1 conjugated SM80s or control SM80s lacking the anti-Iba-1 antibody (containing 1.25 mg Fe, giving an iron dose of ˜5 mg/kg), were intravenously injected via the tail vein and the rats were imaged 24 hours later using T₂w and T₂*w MR sequences according to the scheme shown in FIG. 1.

The following groups of SH rats were used (FIG. 1A): (Group 1, n=11) rats subjected to the MCAO and injected with anti-Iba-1 conjugated SM80 nanoparticles Iba-1-MS80s) 6 days later which were imaged prior to and 24 hours post-injection; (Group 2, n=10) rats subjected to the MCAO and injected with control, non-anti-Iba-1 conjugated SM80s (SM80s) 6 days later which were imaged prior to and 24 hours post-injection; (Group 3, n=9) rats subjected to the MCAO and injected with Iba-1-SM80s 13 days later which were imaged prior to and 24 hours post-injection; (Group 4, n=10) rats subjected to the MCAO and injected with Iba-1-SM80s 27 days later which were imaged prior to and 24 hours post-injection; and (Group 5, n=9) rats subjected to the MCAO and injected with Iba-1-SM80s 6, 13 and 27 days later which were imaged prior to and 24 hours post-injection. (Sham group, n=6) rats, as non-ischemic injury control, injected at 1, 2 and 4 weeks with Iba-1-SM80s which were imaged prior to and 24 hours after each injection. A total of 57 SHRs were used, including two rats which died at day 12 (Group 3) and day 17 (Group 5) after reperfusion during the long-term studies.

Magnetic Resonance Imaging. MRI was performed at 4.7 T with the aid of a Brisker BioSpec 47/40 USR magnet system (Billerica, Mass.) equipped with Avance III electronics^{14, 26-28}. T₂ measurements and T₂w images were taken with twelve 1 mm-thick slices for each rat brain. Gradient-echo T₂*w images and T₂*w measurements were obtained from sixteen slices of 0.5 mm each brain. Both T₂w and T₂*w images were located at rat brain planes within Bregma˜+2.00 to −7.80 mm (The Rat Brain Atlas, Paxinos and Watson 1986).

MRI Data Analysis. The infarct from the MCAO was confined to the right side of the brain and three regions of interest were defined for the ventral and dorsal cortex and the striatum on both the left (L, nonischemic) and right (R, ischemic) hemisphere of the brain (FIG. 1B). The image intensities (R, L) in the T₂w and T₂*w MR images were measured with the aid of ImageJ (https://imagej.nih.gov/ij/index.html) for 350-500 pixels in each of these six regions and used to compute the Right/Left contrast, Cₚ, prior to the injection of the SM80s as Cₚ=(Rᵢ−Lᵢ)/Lᵢ, i∈{1,2,3}. The R/L, contrast alter (Cₐ) injection was similarly defined as Cₐ=(Rⱼ−Lⱼ)/Lⱼ, i∈{1,2,3}. The resulting contrast difference is ΔC=Cₐ−Cₚ. Since the nanoparticle-bound microglia appeared as punctate, hypointense regions in the post-injection T₂*w MR images as a result of the reduction in the T₂* value of the microglia-associated water relative to the surrounding tissue¹⁷, the contrast difference (ΔC) was found to be negative for tissues that had taken up the nanoparticles. We measured a total of 16,980 regions of interest from 55 rats, or an average of 309 measurements from 51 MR image slices per rat. Indicators on images of animal group membership were blinded to the investigators until the measurements were completed.

We observed and corrected for an asymmetry of the intensity between the right and left brain regions even in control rat images due to the inherent radiofrequency inhomogeneity of the surface coil used to obtain the images (Supplementary FIG. 1). We measured this intensity ratio r=I_{R}/I_{L} in the sham rat brains as r=1.20 (n=6) and used it to correct the Left-sided image intensities prior to contrast calculations. Z-scores (contrast to noise ratios) of the punctate hypointensities were computed using measurements of the minimum, M(x,y,z), of the MR signal at its x, y, and z coordinates (z is the slice positions as previously described²¹.

Immunohistology. Perl's Prussian blue and counter staining for detecting Fe⁺-cells were performed on 10 μm formalin-fixed brain sections by Tricore Reference Laboratory (Albuquerque, N. Mex.). For immunohistochemistry, brain sections were stained using anti-Iba-1, anti-TNF-α, anti-IL-1β, anti-IL-10 , anti TGF-β, anti-YM1, and anti-OX-42. (CD 11b).

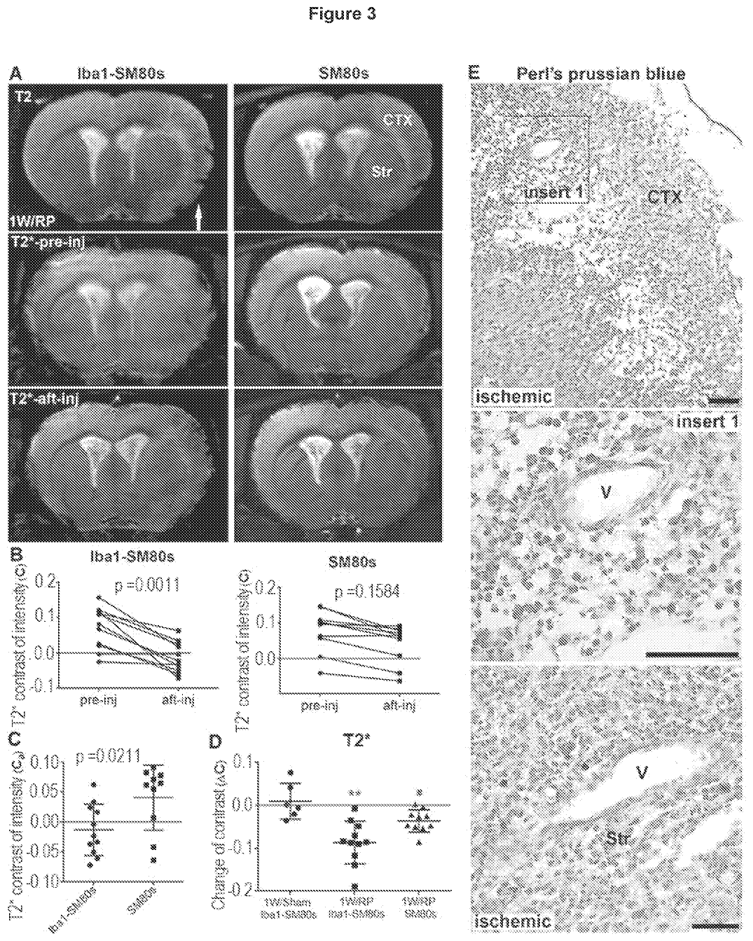

Quantification for colocalization of cytokines with Iba-1. Analysis and quantification of cytokines expressed in Iba-1-positive microglia/macrophages were performed using the colocalization plugin Coloc2 in Fugi-ImageJ (http://fiji.sc/Coloc2) as described in our previous report¹⁴. Li's intensity correlation quotient (ICQ) value, which provides an overall index of colocalization, is distributed between −0.5 and +0.5. Random staining produces an ICQ˜0, while segregated staining gives a negative value (0>ICQ>−0.5) and co-localized staining produces a value of 0<ICQ≤+0.5^{29, 30}. (supplementary FIG. 3). Indicators of animal identity on slides were blinded to the investigator.

Statistics. Unpaired t-tests or one-way Analysis of Variance (ANOVA) were performed for two groups or for multiple group comparisons (with a post-hoc Student-Newman-Keuls test), respectively. Two-way (-factors) ANOVA were performed for group comparisons with time course analysis. In all statistical tests, differences were considered significant when P<0.05. Data are presented as means±SD. Statistical analysis was performed using Prism, version 6.0 (GraphPad Software Incorporated).

### Results

### T₂*w MR Images and Histology One Week after MCAO/RP Show the Iba-1-Conjugated SM80s Only in the Infarcted Region

We first determined that 1 week after MCAO/RP the anti-Iba-1-conjugated SM80s (Iba-1-SM80s), penetrated the BBB, bound to Iba-1⁺-microglia/macrophages and produced hypointensities in the T₂*w MR images of living rat brains (FIG. 1). The initial time point at 1 week was chosen based on studies by ourselves and others showing that activated microglia were observed mainly in the regions within the infarct and peri-infarct areas between 2 and 7 days after transient ischemia^{31, 32}; these reached a peak at 1 week that persisted to 4 weeks¹⁴. To exclude any potential interference from susceptibility variations in the MR images from iron in hemorrhages, animals were imaged twice using a T₂*w MR sequence: the first scan was performed prior to nanoparticle injection (vide supra) where any contribution from hemorrhage-induced susceptibility variations would already be present, while the second MR image set was obtained 24 h after the injection of nanoparticles. The data from the first scan were used as a baseline to compensate for any hemorrhage-related susceptibility variations.

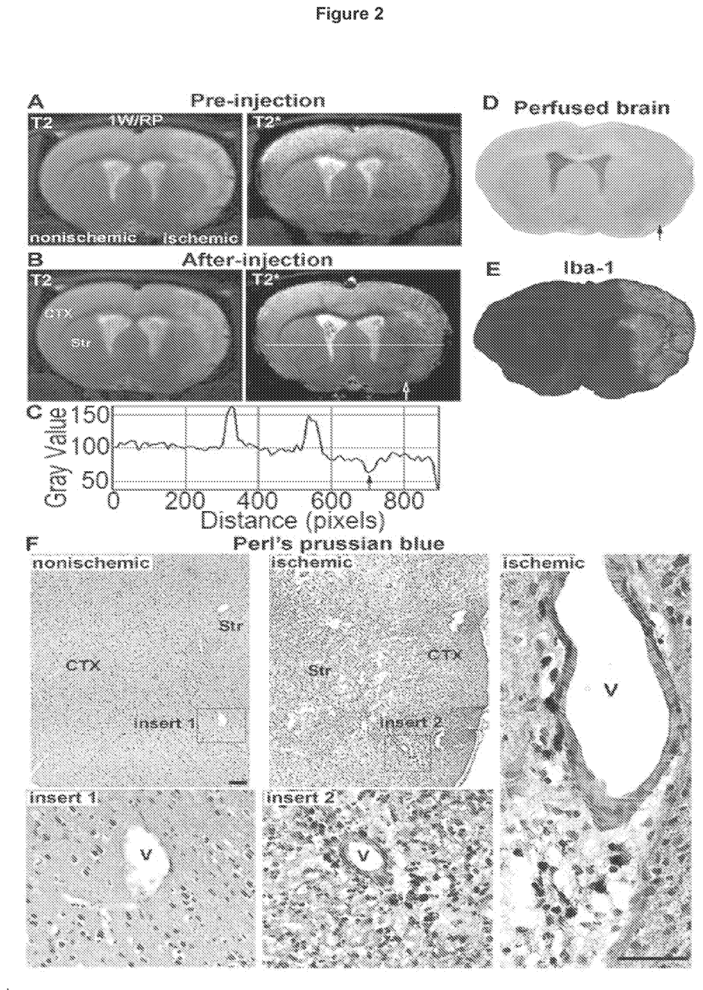

Prior to the injection of the nanoparticles, the infarcted region of the brain displayed a typical pattern of hyperintensities due to cerebral cytotoxic edema in the ischemic hemisphere compared to the nonischemic hemisphere on T₂w and T₂*w MR images (FIG. 2A). This edema lead to an increase of the T₂ and T₂* values in the involved tissues. For example, the T₂ for the contralateral cortex was found to be 60±3 ms (n=24 images), while the T₂ for the ischemic cortex of the brain rose to more than 300 ms. At the same time the T₂* for the nonischemic hemisphere was found to be 50±6 ms (n=24), while the T₂* for the ischemic cortex rose to more than 260 ms.

This background pattern of T₂w MR image hyperintensities continued to be observed. in the T₂w images after the injection of the Iba-1-SM80s (FIG. 2B). The post-injection T₂*w MR images, however, showed prominent, punctate hypointensities in the ischemic hemisphere, suggesting that the injected Iba-1-SM80s were bound to the activated microglia/macrophages clustered within the infarcted region (FIG. 2B, right panel). The nanoparticle-bound microglia/macrophages in the ischemic hemisphere appeared as hypointense regions as a result of the reduction in the T₂* value (FIG. 2C) of the iron bound microglial-associated water relative to the surrounding tissue and nonischemic hemisphere. These T₂*w hypointensities were absent both from the pre-injection images and from the nonischemic hemisphere. The hyperintense regions in the T₂w MR images or the hypointense regions in the T₂*w images corresponded to the lesion regions shown in the post-mortem perfusion-fixed brain (FIG. 2D) and the regions of immunohistochemical staining for activated microglia using anti-Iba-1 antibodies (FIG. 2E) in the same rat. Our data demonstrated that the distribution of the Iba-1-SM480s shown by T₂*w MR imaging in living rat brains were consistent with those of microglia/macrophages labeled by Iba-1 IHC staining on the brain sections.

Perl's Prussian Blue staining of the post-mortem perfused brain sections was utilized to confirm the specific distribution of from the Iba-1-SM80s seen as prominent hypointense regions in the T₂*w MR images in the ischemic hemisphere (FIG. 2F). This showed that the Fe⁺-cells were only observed in the lesion areas of the ischemic hemisphere and that the Iba-1-SM80s were bound to and taken up by cells in locations where the infarct and the Iba-1-SM80s were detected in the T₂*w images (FIG. 2B). Fe⁺-cells were also seen around vessels in the peri-infarct areas (FIG. 2F, right panel), indicating that they indeed crossed the BBB.

### The Specificity of the Iba-1-SM80s was Conferred by the Anti-Iba-1 Antibodies

To show that the specificity of the Iba-1-SM80s was determined by the presence of the surface anti-Iba-1 antibodies, we also prepared control SM80s (denoted as SM80s) that lacked the anti-Iba-1 antibodies, but were identical in all other components. A group (group 2; FIG. 1A) of MCAO/RP SH rats was injected at day 6 with these control SM80s and imaged at day 7 at the presumed peak of Iba-1-positive microglial/macrophage activation¹⁴. A comparison of T₂w images (FIG. 3A, right) from the brains of rats injected with these control SM80s with those from the brains of rats at 7 days after MCAO/RP and injected with the full Iba-1-targeted SM80s (FIG. 3A, left) showed that, even though the infarcts were of similar size in these two groups of rats, significant hypointense regions in the T₂*w MR images were only detected in the ischemic hemisphere from the rats injected with the Iba-1-SM80s, and these were not seen in the images from the brains of rats injected with control SM80s. T₂*w image hypointensities were not seen in the infarcted region either prior to (FIG. 3A, Middle-Right) or after (FIG. 3A, Bottom-Right), the injection of the control SM80s, whereas injection of the full, anti-Iba-1-conjugated SM80s produced image changes similar to those presented in FIG. 2B.

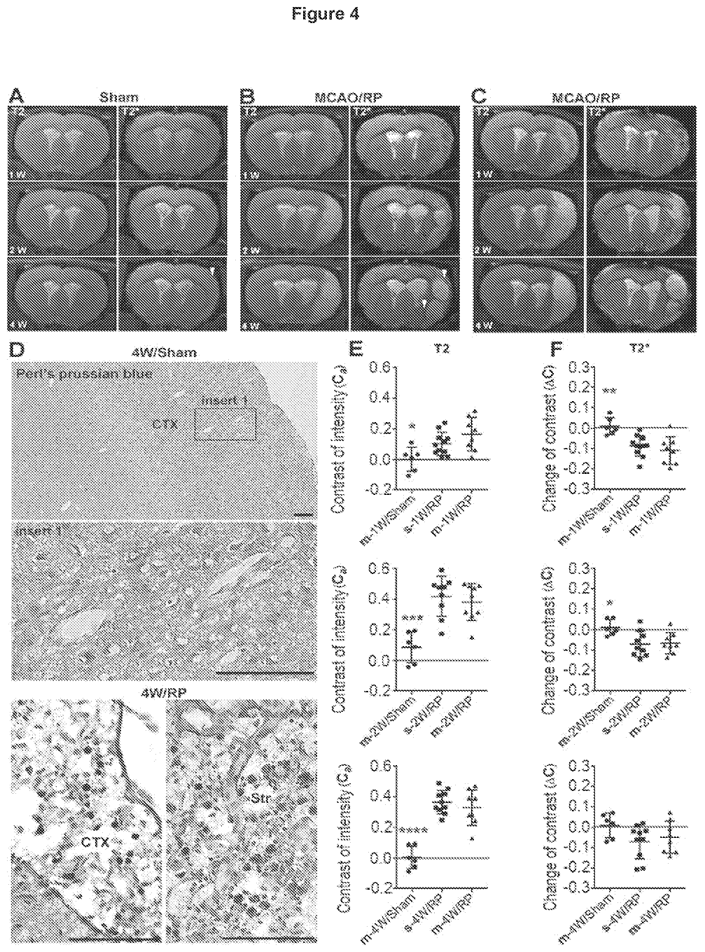

Quantitative contrast measurements (see methods) based on the T₂*w MR images showed that injection of Iba-IMS80s significantly reduced the L/R contrast (Cₐ), in 6 out of 11 rats, compared to the contrast (Cₚ) before injection of the Iba-1MS80s (FIG. 3B, left). No such significant change in contrast was observed when the control SM80s were used (FIG. 3B, right). This result was also supported by the data shown in supplementary FIG. 4 where the T₂*w image intensities between nonischemic and ischemic hemispheres were compared. Comparison of the post-injection contrast values (Cₐ) between the Iba-1-SM80s and the control SM80s (FIG. 3C) gave additional support for this result.

Furthermore, we measured the contrast change (ΔC=Cₐ−Cₚ) in T₂*w MR images (FIG. 3D). The mean of the T₂*w contrast change was essentially zero (ΔC=+0.009) in the sham rats injected with the Iba-1-SM80s at 1 week, indicating a lack of uptake of the Iba-1-SM80s. Injection of the Iba-1-SM80s into rats 7-day after MCAO/RP resulted in a robust mean negative contrast change of ΔC=−0.087, suggesting significant uptake of the Iba-1-SM80s in the infarcted region compared to the sham rats (p=0.0011). Significant uptake of the control SM80s (ΔC−0.036) in the infarcted region 7 days after MCAO/RP was detected comparing to the sham rats (p=0.0164), reflecting some non-specific phagocytosis of the control SM80s by active microglia/macrophage. However, the uptake of the control SM80s was significantly lower than that of Iba-1-MS80s in the infarcted region (p=0.0099). Accordingly, the injection of control SM80s resulted in only a few Fe⁺ cells in the ischemic hemisphere (FIG. 3E), which differed markedly from the strong Perl staining seen in the brain injected with Iba-1-SM80s at 1 week after MCAO/RP (FIG. 2F).

### Longitudinal MRI after Multiple Injections of Iba-1-SM80s Showed that They Did Not Accumulate in the Brain Tissue

To determine whether the FePt nanoparticles accumulated in the brain over time, we imaged groups of rats (including sham rats) that received injections of Iba-1-SM80s at 1, 2 and 4 weeks (for a total of 3 injections) and compared the results with rats that received single injections of Iba-1-SM80s at either 1, 2 or 4 weeks, respectively. T₂w and T₂*w MR images of the brains in sham rats obtained after the injections of Iba1-SM80s at 1, 2, and 4 weeks showed an absence of both edema and image hypointensities (FIG. 4A). On the other hand, we observed numerous punctate hypointensities in the T₂*w images obtained after the injection of Iba1-SM80s which peaked 1 week after MCAO/RP, and diminished from 2 to 4 weeks after MCAO/RP, and mostly surrounded the core infarct areas (FIG. 4B). This result indicated that the nanoparticles did not accumulate in the tissue over the course of this study. This result was also congruent with imaging performed on animals who received only a single injection of Iba1-SM80s at 1, 2, or 4 weeks, respectively, where significant T₂*w hypointensities were only observed at the one week time point (FIG. 4C. upper right). Furthermore, post-mortem Perl's Prussian Blue staining of the brains from rats that received 3 injections of Iba1-SM80s showed no Fe⁺-cells in the sham brain, while only a slight residuum of Perl's Fe+ cells were seen in the peri-infarct areas in the striatum in the 4-week MCAO/RP brain (FIG. 4D).

In order to place these qualitative results onto a quantitative basis, we measured the T₂w image contrast Cₐ (FIG. 4E) and T₂*w MR image contrast change ΔC (FIG. 4F) at 1, 2, and 4 weeks after either single or multiple injections of Iba1-SM80s into groups of sham and. MCAO/RP rats. Compared with sham rat brains, T₂w image contrast measurements showed a significant increase of the Cₐ values in rat brains receiving either single (s) or multiple (m) injections of Iba1-SM80s at all three time points, due to the continued development of edema in the infarcted regions (FIG. 4E). There were no significant contrast differences between single and multiple injections groups at any time point. Measurements of the contrast differences (ΔC=Cₐ−Cₚ) from the T₂*w MR images of the groups injected with Iba1-SM80s showed no contrast difference (ΔC=0) in the brains from the sham rats injected with Iba1-SM80s for any point from 1 to 4 weeks, independent of the number of injections (FIG. 4F). Negative ΔC values were found in the MCAO/RP group of rats at all time points investigated, while significant negative ΔC values were observed compared to sham rats independent of the number of injections at 1 week and 2 weeks. In addition, we also measured the post injection contrast (Cₐ) in T₂*w images and found that a significant difference of the Cₐ was only seen when sham and 1-week MCAO/RP groups were compared (supplementary FIG. 5). Importantly, the results show that single or multiple injections produced the same T₂w contrast (Cₐ), T₂*w contrast (Cₐ), and T₂*w contrast changes (ΔC), at each time point.

### Longitudinal Monitoring of Microglial Activation MCAO/RP with Iba1-SM80-Enhanced MR Imaging and Histochemistry

To determine the longitudinal time-course of stroke-induced inflammation after ischemia and to map the specific changes in microglial/macrophage activation, we further compared the Iba-1-SM80 distribution measured from T₂*w MR images with those from the histological analyses for Iba-1⁺-microglia/macrophages at 1, 2, and 4 weeks after MCAO/RP. T₂*w MR images of the rat brains 24 hours after single Iba-1-SM80 injections into either sham, or MCAO/RP rats showed the locations of the SM80s as punctate, hypointense areas in the infarct and peri-infarct regions (FIG. 5A). After the several MR images were obtained, the rats were sacrificed and the brains were sectioned for histological studies. The regions of microglial/macrophage activation revealed by Iba-1 antibody staining (FIG. 5A, right) closely coincided with the regions of edema and SM80 uptake in the T₂w* MR images (FIG. 5A, left) at all three time points. No hypointense areas and activated microglia/macrophages were observed in sham rat brains. The results from both imaging techniques indicated that the activation of the microglia in response to MCAO/RP peaked at 1 week, and then diminished significantly from 2 to 4 weeks when activated microglia/macrophages were only seen in the peri-infarct areas. The same time course of changes was found when comparing the densities of Perl's stained (Fe⁺) cells and Iba-1⁺-microglia in the same rat brain tissues (FIG. 5B). Various morphological shapes of Fe⁺-cells and Iba-1⁺-microglia were seen at different time points (FIG. 5B, inserts). At 1 to 2 weeks, the vast majority of Fe⁺-cells and Iba-1⁺-microglia cells in the adjacent brain sections were round in shape in the core infarct areas, while both the Fe⁺-cells and Iba-1⁺-microglia with extended processes appeared in the peri-infarct areas by 4 weeks. This observation suggests that the Fe⁺-cells are active microglia/macrophages that took up the Iba-1-SM80s.

Since no differences were detected in the T₂w and T₂*w MR contrast data between single and multiple SM80 injections (FIG. 4 and supplementary FIG. 5), we combined the MRI contrast data from both the single and the multiple injection groups at each time point to determine the longitudinal time-course of stroke-induced inflammatory changes. The marked hyperintensities observed in T₂w MR images in the MCAO/RP rat groups (FIG. 4B and C) reflected edema formation in the ischemic hemispheres after MCAO/RP. Measurements of T₂w MR, image contrast (Cₐ) demonstrated the development of edema over the 4 weeks after MCAO/RP compared with sham rats (FIG. 5C left). However, no significant contrast differences (ΔC) were determined using T₂w images (FIG. 5C, right), because the edema was the same both before and after the injection of the Iba-1-SM80s, and this hyperintense background subtracted out. The small contrast differences determined using T₂w images suggested a small effect of the nanoparticles on the tissue T₂.

Because the measured r₂* relaxivity of the SM80s (853 Hz/mM) was almost three-fold greater than their r₂ relaxivity (300 Hz/mM)¹⁷, we expected that T₂*w MR images would show the largest effect of the SM80s. T₂*w MR image contrast (Cₐ), and contrast changes (ΔC) reflected the time-dependent presence of the Iba-1-SM80s (FIG. 5D) with the maximum effect observed at one week post-stroke. Both of these measures of neuroinflammation diminished with time from week 1 to week 4, due to the decreased specific binding of Iba-1-SM80s, in concert with the observed decrease of the Iba-1⁺-microglia/macrophages over time after MCAO/RP (FIG. 5B). Notably, the T₂*w MRI contrast change (ΔC, FIG. 5D, right) demonstrated the most robust reflection of the time-dependent changes of microglial activation, and the ΔC values for the sham rats were consistently zero.

### Three-Dimensional Mapping of the SM80s within the Brain Parenchyma

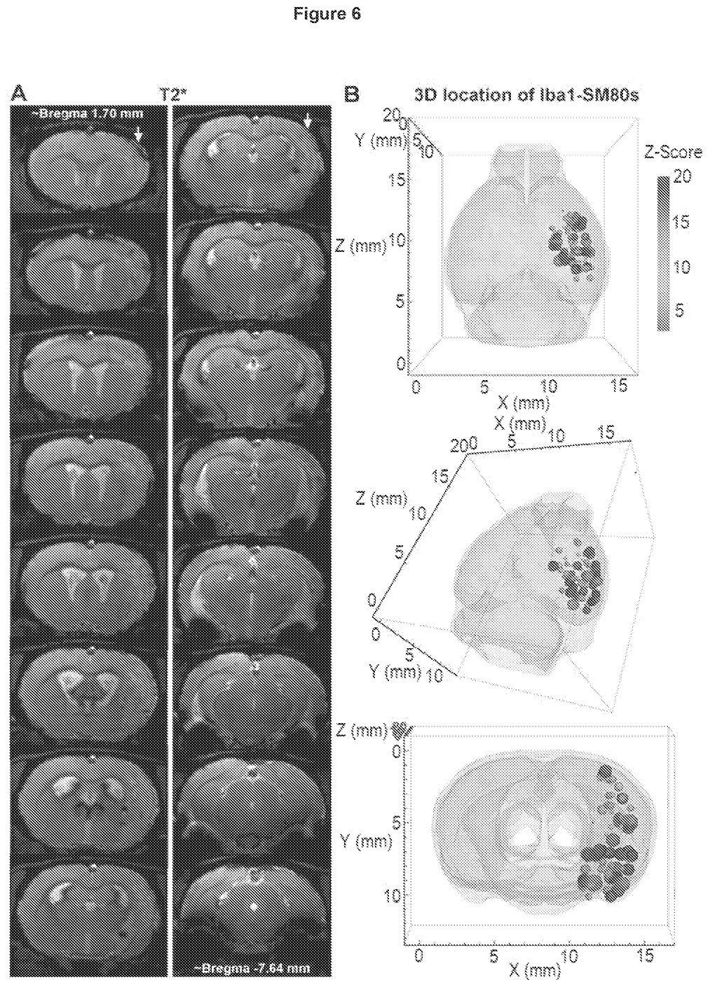

Prior studies of the response of microglia to stroke have indicated that microglia in the peri-infarct area have different patterns of microglial activation from those found in the ischemic core, and the distribution of the microglia was one of the critical factors that determined the microglial phenotype⁷. MR images are quantitative maps of these tissue relaxation characteristics in 3-dimensions. The SM80s shorten the transverse relaxation time, T₂*, of water. We used the T₂*w MR images to generate 3D maps of the distribution of the activated microglia. A representative set of T₂*w MR images from a rat brain at 1 week after MCAO/RP displayed the punctate regions of hypointensity (FIG. 6A) in the ischemic hemisphere, indicating that the Iba1-SM80s bound exclusively to the activated microglia. Measurements of the positions and Z-scores (See Methods) of the hypointense voxels were used to generate a 3-dimensional map of the Iba-1-SM80s, and hence the activated microglia/macrophages in the post-MCAO brain (FIG. 6B). The results showed that the magnetic nanoparticles were found only within the brain area affected by the ischemic intervention located in the brain regions from +1.70 to −7.64 mm with respect to the Bregma.

### Changes of Expression of Pro- and Anti-Inflammatory Cytokines in the Active Microglia/Macrophages from One to Four Weeks after MCAO/RP

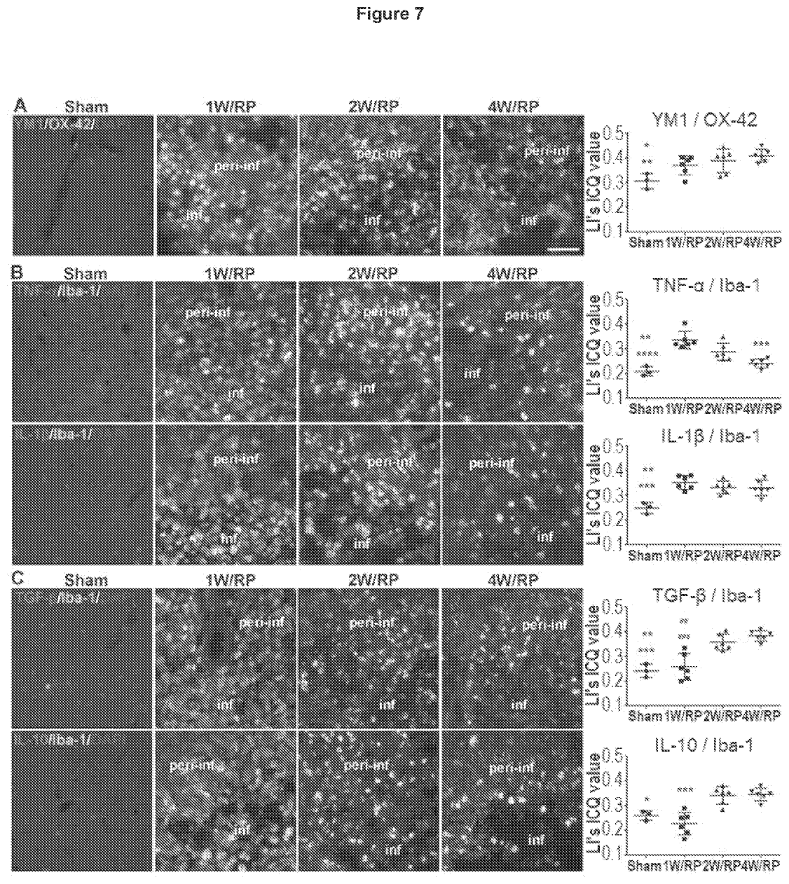

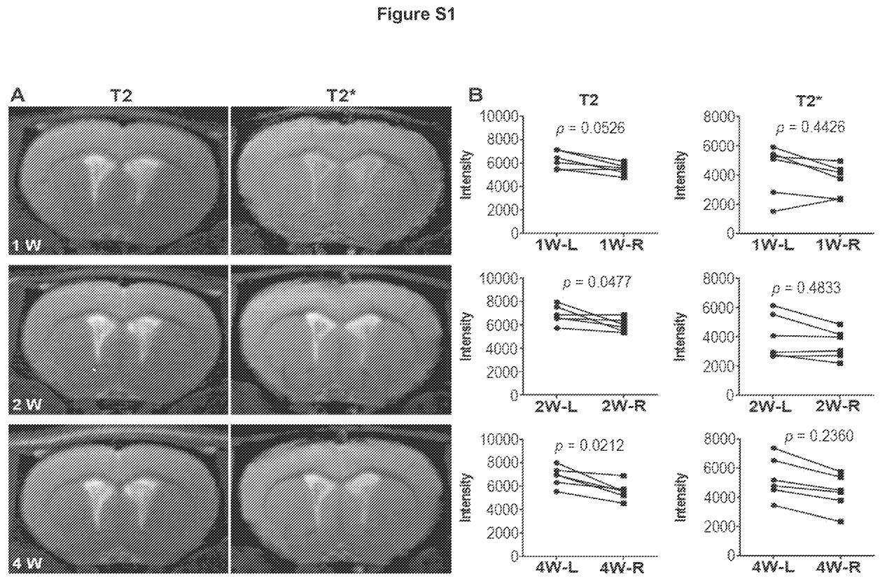

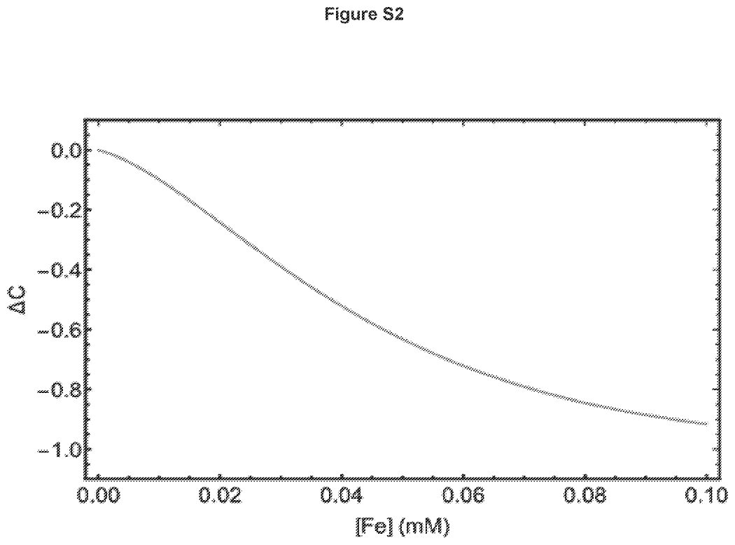

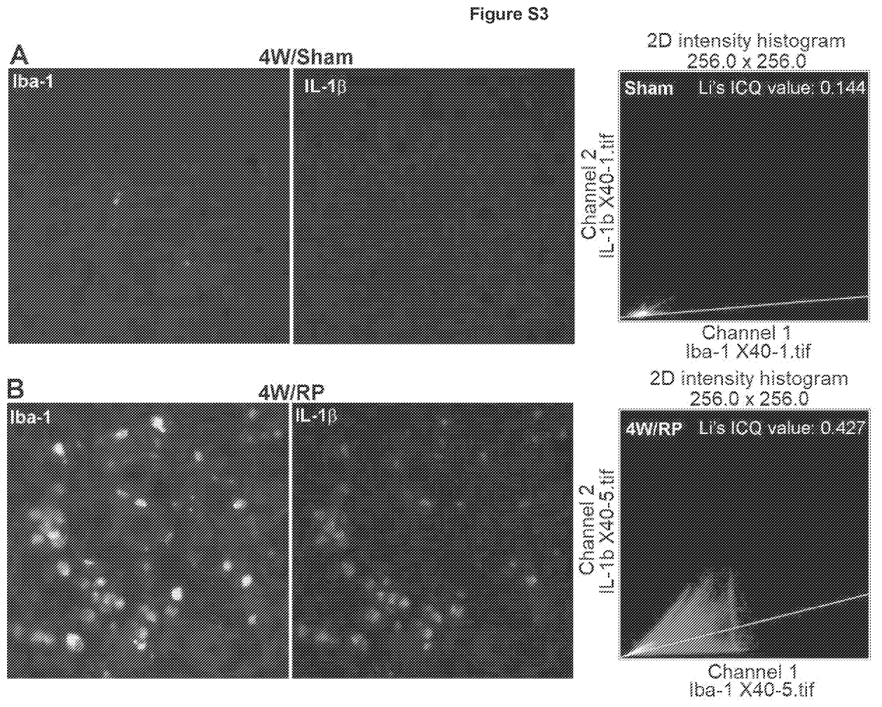

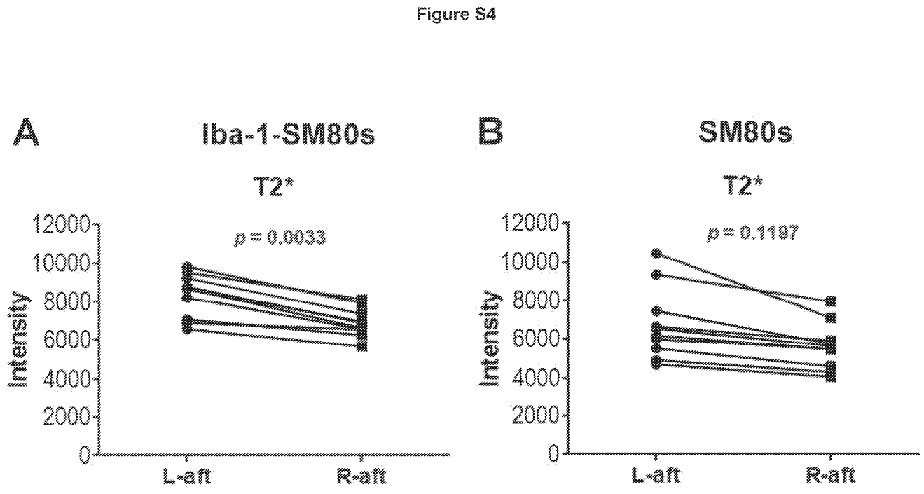

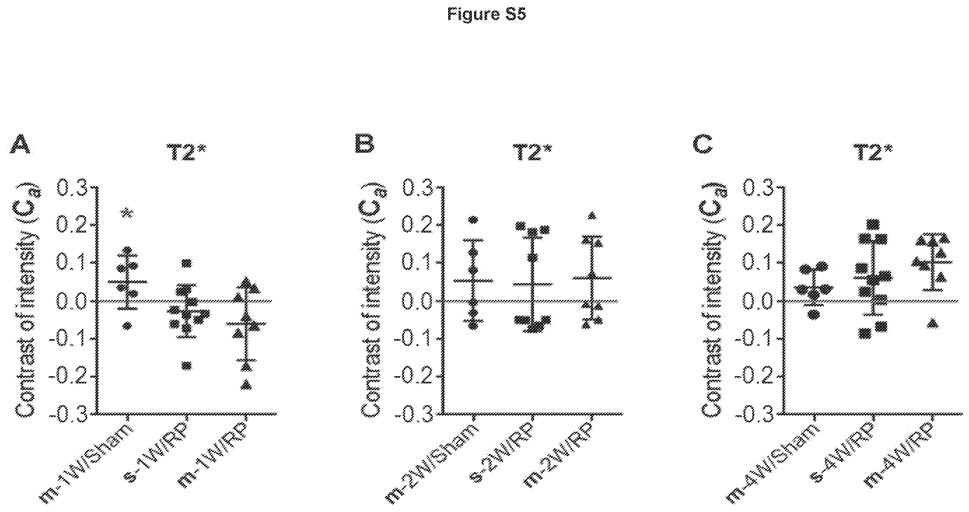

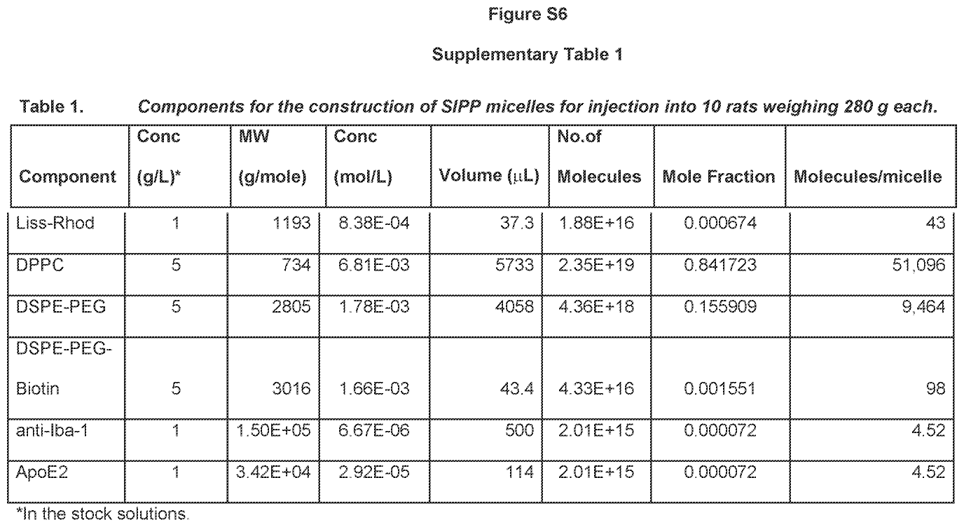

To correlate the changes of the distribution of the SM80s in ischemic rat brains observed by T₂*w MRI with the time-dependence of the switch of the microglial/macrophage immunophenotype from its pro- to its anti-inflammatory state, we performed double-immunohistochemistry (IHC) to detect the expression of pro- or anti-inflammatory cytokines in Iba-1⁺- or OX-42 (CD11b)⁺-microglia/macrophages in ischemic brains. After the MRI scans were finished, the rat brains were prepared for histological studies. We first examined the expression of YM1, a marker of regulatory/anti-inflammatory microglia/macrophages, in OX-42⁺-microglia/macrophages. Around the border of the infarct core and in the peri-infarct areas where the SM80s were detected by T₂*w imaging, we found increasing expression of YM1 in microglia from 1 week to 4 weeks after MCAO/RP (FIG. 7A, left). We quantified the co-localization of YM1 and OX-42 using the colocalization plugin Coloc2 in Fiji-ImageJ (see Methods and Supplementary FIG. 3 for definition). The large values of Li's ICQ showed that the co-localization increased at 2 and 4 weeks after MCAO/RP, compared to sham rats, suggesting that shifting of the microglial/macrophage activation from the pro-inflammatory to the anti-inflammatory state occurred during 2 and 4 weeks after MCAO/RP (FIG. 7A, right). We next examined the expression of four cytokines in the microglia/macrophages within the infarct and peri-infarct areas. The pro-inflammatory cytokines TNF-α and IL-1β were expressed by pro-inflammatory microglia/macrophages, anti-inflammatory cytokines TGF-β and IL-10 were expressed by regulatively-activated microglia/macrophages^{1, 3, 7-9, 33-36}. Compared to sham brains, significantly increased expression of TNF-α and IL-1β in active microglia/macrophages was found 1 to 2 weeks after MCAO/RP (FIG. 7B). Decreased expression of TNF-α was seen from week 2, which reached a significant reduction at 4 weeks compared to 1 week (FIG. 7 B, upper). A slight decrease of IL-1β expression was seen in microglia at 2-4 weeks (FIG. 7B, bottom), but the double-IHC staining showed that most of the IL-1β was expressed by the microglia located in the infarct area. On the other hand, significantly increased expression of TGF-β and 1L-10, the anti-inflammatory cytokines, was seen at weeks 2 to week 4, compared to the sham and 1-week MCAO/RP groups (FIG. 7C). These results were temporally and spatially consistent with the changes of YM1 expression in active microglia in response to stroke-induced brain injury.

### Discussion

The inventors have demonstrated that MRI, enhanced with unique anti-Iba-1-conjugated iron-platinum nanoparticles, was able to reveal the spatial distribution and longitudinal time-course of stroke-induced inflammation and to reveal the specific, localized changes in microglial/macrophages activation. A comparison of the post-mortem histological data with the location of lesion areas, determined in vivo from T₂w anatomic MR imaging and from the sites of microglial/macrophages activation from T₂*w MR imaging, showed that the nanoparticle-enhanced MR images reflected the change of the distribution of Iba-1⁺-microglia/macrophages over time from 1 to 4 weeks after stoke. Our MRI and histological data also correlated with time-dependent measurements of pro- and anti-inflammatory cytokines as the predominant phenotypic transformation in the active microglia/macrophages after stoke.

Treatments that target pro-inflammatory microglia/macrophages and treatments that enhance regulatory/anti-inflammatory features of microglia/macrophages would provide attractive therapeutic opportunities for cerebral stroke, especially, for promoting neurovascular remodeling during stroke recover^{1, 3, 4, 10, 11,15,16}. Therefore, it is vital to delineate the time course of the underlying changes in microglial/macrophage activation occurring during stroke recovery^{2, 37, 38}. Although the use of peripheral and imaging biomarkers has improved the in vivo diagnosis of stroke, it would be of great clinical relevance to non-invasively monitor the longitudinal development of inflammation in the brain of stroke subjects. Since MRI is widely used in clinical practice, an MRI method for imaging the inflammatory response in vivo would support investigations comparing the efficacy of treatments²¹. We paved the way for the application of nanoparticle-enhanced MRI to stroke by building on our earlier studies where novel FePt nanoparticles was used to measure neuroinflammation in 3×transgenic Alzheimer's mice²⁴. In the current study, these FePt nanoparticles were incorporated into phospholipid micelles (SM80s), which were tailored to minimize interaction with the reticuloendothelial system in order to extend their lifetime in the blood and to avoid the buildup of a protein corona by means of their polyethyleneglycol-coated surfaces. The addition of a polysorbate-80³⁹⁻⁴² coating and apoE2⁴³ provided a mechanism by which the nanoparticles were transported through the BBB via interaction with the LDL receptors in the brain. In order to specifically reveal neuroinflammation the SM80s were targeted to activated microglia/macrophages by conjugating anti-Iba-1 antibodies.

We measured the MRI contrast between the nonischemic and ischemic hemispheres of the brain, and the MRI contrast changes prior to and after nanoparticle injection to evaluate the activation of microglia in rat brains up to four weeks after MCAO/RP. The injection of Iba-1-SM80s into sham-operated rats produced no significant changes in the MR contrast from one to four weeks after surgery. On the other hand, the injection of Iba-1-SM80s into rats subjected to the MCAO/RP resulted in significant specific alterations in the MRI contrast at one week, which became less pronounced by two to four weeks. This time course of MR contrast changes paralleled the histological features shown by immunochemical staining for activated microglia/macrophages using anti-Iba-1 antibodies and optical microscopy. By comparing the T₂*w MR images with those from the histological analyses, we demonstrated that the regions of edema and nanoparticle distribution in the MR images coincided with the regions of microglial/macrophage activation over the time course examined. These results provided comprehensive information with respect to how MRI imaging of labeled microglia/macrophage reflects real-time microglial/macrophage activation occurring in the ischemic brain.

We found various morphological shapes and locations of the Fe⁺-cells at different time points over the four weeks of reperfusion. The round shapes of the Fe⁺-cells found predominantly in the core infarct areas from one to two weeks were similar to the brain resident microglia and blood-derived macrophages found after stroke^{14, 22, 44}. The Fe⁺-microglia/macrophages with extended processes were similar to the new population of active, anti-inflammatory cytokine-expressing microglia/macrophages in the peri-infarct areas four weeks after stroke as we reported before¹⁴. The pro-inflammatory response of microglia/macrophages includes increased expression of the cytokines TNF-α and IL-1β, while in the regulatory/homeostatic phase, expression switches to the anti-inflammatory or reparative cytokines, such as IL-10 and IGF-β, and their co-stimulatory protein^{3, 33, 44}. Our histological analysis demonstrated that a switch of the microglial immunophenotype occured from weeks 2 to 4 after MCAO/RP. This time-dependent switch was coincident with the maximum microglial expression of TNF-α and IL-1β, the pro-inflammatory cytokines, at one week, while increased microglial expression of YM1, H-10, and TGF-β, the anti-inflammatory cytokines, occurred from weeks two to four. Importantly, the maximum negative ΔC value from T₂*w MR imaging seen at one week coincided with the maximum expression of the pro-inflammatory markers markers, TNF-α and IL1β in the activated microglia in the region of the infarct, while decreasing negative ΔC values coincided with the anti-inflammatory microglia/macrophages during weeks two to four. These findings suggested that combining targeted magnetic nanoparticles with MR imaging constituted a novel approach to non-invasively detect and monitor the temporal profile of inflammation and microglial activation in living animal^{45, 46}.

The inventors found several factors that interfered with the quantification of the SM80s detected by T₂w and T₂*w imaging. One of the factors was that the hypointensities in the T₂*w MR images in the infarcted region of the brain appeared against a hyperintense background due to the cytotoxic edema in the core infarct areas, particularly at later stages after MCAO/RP. This led to the finding of, for example, a positive R/L contrast value Cₐ=(I_{R}−I_{L})/I_{L} in the T₂*w MR images from the group of 4-week MCAO/RP rats, which did not correctly reflect the obvious presence of hypointense regions surrounding core infarct areas that contained Iba-1⁺-active microglia that were confirmed by histological staining. To exclude this edema interference, we employed the image contrast change (ΔC=Cₐ−Cₚ) obtained pre-(Cₚ) and after(Cₐ)-injection of MS80s. The contrast change provided a quantitative measure of the delivery of the nanoparticles to the infarcted tissue consistent with those from both T2*w MRI and histology. This technique produced a contrast change value of ΔC=0.00 for the sham rats.

In the preliminary studies, the inventors observed that MCAO/RP occasionally induced hemorrhage in the rat model of MCAO. The iron in hemorrhages may be expected to enhance the T₂*w image and interfere with the evaluation of the location of activated microglia. To circumvent this potential issue, we subjected the animals to two MRI scans at each designated time point: the first of T₂w and T₂*w scans was performed prior to nanoparticle injection, while the second was performed 24 hours after nanoparticle injection. The data from the first scans were used as a baseline image to remove any potential effect of a hemorrhage, allowing the presence and distribution of nanoparticle-bound microglia to be accurately determined. Unexpectedly, this experimental design turned out to be necessary for the MRI data evaluation to exclude the interference of stroke-induced brain edema.

One final concern in this work was the question of whether repeated injections of SM80s would build up in the brain and render useless our attempts to follow the time course of microglial activation in individual rats. In one of our related, but separate (unpublished) studies on breast tumor xenografts in mice, we found that SPIONs injected via tail vein were metabolized and no longer provided negative tumor contrast 96 hours post-injection. However, it was still possible that the cerebral dynamics of SM80s were sufficiently different that microglia retained them for more than the one-week minimum between injections. MR images from the sham rats showed no such retention, even though they received up to 3 injections at one, two, and four weeks. The MRI data obtained from the MCAO/RP rats also showed no retention indicating that iron from the FePt cores was metabolized within the week between injections. In addition, the histological analysis showed that multiple injections of SM80s did not lead to the accumulation of Fe within the brain tissue.

These initial studies revealed the potential for the use of FePt magnetic nanoparticles when using MRI imaging to delineate the regions of microglial activation within the infarcted region of the stroke brain. We believe that our current positive results support a future effort devoted to the exploration of the characteristics of the FePt particles. In particular, it would appear that a concerted effort would be desirable to explore the composition of the SIPP micelles with the goal of optimizing their penetration of the BBB and their delivery to brain regions whose perfusion was compromised in stroke. We have yet to examine the relationship between antibody surface density and BBB penetration. This also applies to the question of the optimal lipid composition of the micelles and the density of the Iba-1 on their surfaces. Future studies will also utilize this novel approach to monitor the neuroinflammatory and recovery progression in animal model of stroke with therapeutic interventions.

### REFERENCES

1. Yong H Y F, Rawji K S, Ghorbani S, Xue M, Yong V W. The benefits of neuroinflammation for the repair of the injured central nervous system. Cellular & molecular immunology 2019; 16(6): 540-546.
2. Ceulemans A G, Zgave T, Kooijman R, Hachimi-Idrissi S, Sarre S, Michotte Y. The dual role of the neuroinflammatory response after ischemic stroke: modulatory effects of hypothermia. J Neuroinflammation 2010; 7: 74.
3. Bai Q, Xue M, Yong V W. Microglia and macrophage phenotypes in intracerebral hemorrhage injury: therapeutic opportunities. Brain: a Journal of neurology 2020.
4. Schwartz M, Deczkowska A. Neurological Disease as a Failure of Brain-Immune Crosstalk: The Multiple Faces of Neuroinflammation. Trends in immunology 2016; 37(10): 668-679.
5. Pimenova A A, Marcora E, Goate A M. A Tale of Two Genes: Microglial Apoe and Trem2. Immunity 2017; 47(3): 398-400.
6. Krasemann S, Madore C, Cialic R, Baufeld C, Calcagno N, El Fatimy R et al. The TREM2-APOE Pathway Drives the Transcriptional Phenotype of Dysfunctional Microglia in Neurodegenerative Diseases. Immunity 2017; 47(3): 566-581 e9.
7. Taylor R A, Sansing L H. Microglial responses after ischemic stroke and intracerebral hemorrhage. Clinical & developmental immunology 2013; 2013: 746068.
8. Lampron A, Elali A, Rivest S. Innate immunity in the CNS: redefining the relationship between the CNS and Its environment. Neuron 2013; 78(2): 214-32.
9. Jin R, Yang G, Li G. Inflammatory mechanisms in ischemic stroke: role of inflammatory cells. Journal of leukocyte biology 2010; 87(5): 779-89.
10. Gelderblom M, Leypoldt F, Steinbach K, Behrens D, Choe C U, Siler D A et al. Temporal and spatial dynamics of cerebral immune cell accumulation in stroke. Stroke; a journal of cerebral circulation 2009; 40(5): 1849-57.
11. Gulke E, Gelderblom M, Magnus T. Danger signals in stroke and their role on microglia activation after ischemia. Therapeutic advances in neurological disorders 2018: 11: 1756286418774254.
12 Cotrina M L, Lou N, Tome-Garcia J, Goldman J, Nedergaard M. Direct comparison of microglial dynamics and inflammatory profile in photothrombotic and arterial occlusion evoked stroke. Neuroscience 2017; 343: 483-494.
13. Thiel A, Heiss W D. Imaging of microglia activation in stroke. Stroke; a journal of cerebral circulation 2011; 42(2): 507-12.
14. Yang Y, Salayandia V M, Thompson J F, Yang L Y, Estrada E Y, Yang Y. Attenuation of acute stroke injury in rat brain by minocycline promotes blood-brain barrier remodeling and alternative microglia/macrophage activation during recovery. J Neuroinflammation 2015; 12: 26.
15. Yenari M A, Kauppinen T M, Swanson R A. Microglial activation in stroke: therapeutic targets. Neurotherapeutics: the journal of the American Society for Experimental Neurotherapeutics 2010; 7(4): 378-91.
16. Taylor R M, Huber D L, Monson T C, Ali. A M, Bisoffi M, Sillerud L O. Multifunctional iron platinum stealth immunomicelles: targeted detection of human prostate cancer cells using both fluorescence and magnetic resonance imaging. Journal of nanoparticle research: an interdisciplinary forum for nanoscale science and technology 2011; 13(10): 4717-4729.
17. Taylor R M, Huber D L, Monson T C, Esch V, Sillerud L O. Structural and Magnetic Characterization of Superparamagnetic Iron Platinum Nanoparticle Contrast. Agents for Magnetic Resonance Imaging. Journal of vacuum science and technology. B, Nanotechnology & microelectronics: materials, processing, measurement, & phenomena: JVST B 2012; 30(2): 2C101-2C1016.
18. Sillerud L O. Quantitative [Fe]MRI determination of the dynamics of PSMA-targeted SPIONs discriminates among prostate tumor xenografts based on their PSMA expression. Journal of magnetic resonance imaging: JMRI 2018.
19. Sillerud L O. Quantitative [Fe]MRI of PSMA-targeted SPIONs specifically discriminates among prostate tumor cell types based on their PSMA expression levels. International journal of nanomedicine 2016; 11: 357-71.
20. Taylor R M, Severns V, Brown D C, Bisoffi M, Sillerud L O. Prostate cancer targeting motifs: expression of alphanu beta3, neurotensin receptor 1, prostate specific membrane antigen, and prostate stem cell antigen in human prostate cancer cell lines and xenografts. The Prostate 2012; 72(5): 523-32.
21. Sillerud L O, Solberg N O, Chamberlain R, Orlando R A, Heidrich J E, Brown D C et al. SPION-enhanced magnetic resonance imaging of Alzheimer's disease plaques in AbetaPP/PS-1 transgenic mouse brain. Journal of Alzheimer's disease: JAD 2013; 34(2): 349-65.
22. Ito D, Tanaka K, Suzuki S, Dembo T, Fukuuchi Y. Enhanced expression of Iba1, ionized calcium-binding adapter molecule 1, after transient focal cerebral ischemia in rat brain. Stroke; a journal of cerebral circulation 2001; 32(5): 1208-15.
23. Taylor R M, L O. Paclitaxel-loaded iron platinum stealth immunomicelles are potent MRI imaging agents that prevent prostate cancer growth in a PSMA-dependent manner. International journal of nanomedicine 2012; 7: 4341-52.
24. Tafoya M A, Madi S, Sillerud L O. Superparamagnetic nanoparticle-enhanced MRI of Alzheimer's disease plaques and activated microglia in 3×transgenic mouse brains: Contrast optimization. Journal of magnetic resonance imaging: JMRI 2017; 46(2): 574-588.
25. Kilkenny C, Browne W, Cuthill I C, Emerson M, Altman D G, National Centre for the Replacement R et al. Animal research: reporting in vivo experiments—the ARRIVE guidelines. Journal of cerebral blood flow and metabolism: official journal of the international Society of Cerebral Blood Flow and Metabolism 2011; 31(4): 991-3.
26. Yang Y, Kimura-Ohba S, Thompson J F, Salayandia V M, Cosse M, Raz L et al. Vascular tight junction disruption and angiogenesis in spontaneously hypertensive rat with neuroinflammatory white matter injury. Neurobiology of disease 2018; 114: 95-110.
27. Yang Y, Thompson J F, Taheri S, Salayandia V M, McAvoy T A, Hill J W et al. Early inhibition of MMP activity in ischemic rat brain promotes expression of tight junction proteins and angiogenesis during recovery. Journal of cerebral blood flow and metabolism: official journal of the International Society of Cerebral Blood Flow and Metabolism 2013; 33(7): 1104-14.
28. Yang Y, Yang L Y, Orban L, Cuylear D, Thompson J, Simon B et al. Non-invasive vagus nerve stimulation reduces blood-brain barrier disruption in a rat model of ischemic stroke. Brain stimulation 2018; 11(4): 689-698.
29. Li Q, Lau A, Morris T J, Guo L, Fordyce C B, Stanley E F. A syntaxin 1, Galpha(o), and N-type calcium channel complex at a presynaptic nerve terminal: analysis by quantitative immunocolocalization. The Journal of neuroscience: the official journal of the Society for Neuroscience 2004; 24(161): 4070-81.
30. Bolte S, Cordelieres F P. A guided tour into subcellular colocalization analysis in light microscopy. Journal of microscopy 2006; 224(Pt 3): 213-32.
31. Rojas S, Martin Arranz M J, Pareto D, Purroy J, Verdaguer E et al. Imaging brain inflammation with [(11)C]11195 by PET and induction of the peripheral-type benzodiazepine receptor after transient focal ischemia in rats. Journal of cerebral blood flow and metabolism: official journal of the International Society of Cerebral Blood Flow and Metabolism 2007; 27(12): 1975-86.
32. Schroeter M Dennin M A, Walberer M, Backes H, Neumaier B, Fink G R et al. Neuroinflammation extends brain tissue at risk to vital peri-infarct tissue: a double tracer [11C]PK11195- and [18F]FDG-PET study. Journal of cerebral blood flow and metabolism: official journal of the International Society of Cerebral Blood Flow and Metabolism 2009; 29(6): 1216-25.
33. Colton C A, Heterogeneity of microglial activation in the innate immune response in the brain. Journal of neuroimmune pharmacology: the official journal of the Society On Neuroimmune Pharmacology 2009; 4(4): 399-418.
34. Giunti D, Parodi B, Cordano C, Uccelli A, Kerlero de Rosbo N. Can we switch microglia's phenotype to foster neuroprotection? Focus on multiple sclerosis. Immunology 2013.
35. Gordon S, Martinez F O. Alternative activation of macrophages: mechanism and functions. Immunity 2010; 32(5): 593-604.
36. Varin A, Mukhopadhyay S, Herbein G, Gordon S. Alternative activation of macrophages by IL-4 impairs phagocytosis of pathogens but potentiates microbial-induced signalling and cytokine secretion. Blood 2010; 115(2): 353-62.
37. Eyo U B, Dailey M E. Microglia: key elements in neural development, plasticity, and pathology. Journal of neuroimmune pharmacology: the official journal of the Society on NeuroImmune Pharmacology 2013; 8(3): 494-509.
38. Iadecola C, Anrather J. The immunology of stroke: from mechanisms to translation. Nature medicine 2011; 17(7): 796-808.
39. Ambruosi A, Khalansky A S, Yamamoto H, Gelperina S E, Begley D J, Kreuter J. Biodistribution of polysorbate 80-coated doxorubicin-loaded [14C]-poly(butyl cyanoacrylate) nanoparticles after intravenous administration to glioblastoma-bearing rats. Journal of drug targeting 2006; 14(2): 97-105.
40. Ramge P, Unger R E, Oltrogge J B, Zenker D, Begley D, Kreuter J et al. Polysorbate-80 coating enhances uptake of polybutylcyanoacrylate (PBCA)-nanoparticles by human and bovine primary brain capillary endothelial cells. The European Journal of neuroscience 2000; 12(6): 1931-40.
41. Yusuf M, Khan M, Khan R A, Maghrabi I A, Ahmed B. Polysorbate-80-coated, polymeric curcumin nanoparticles for in vivo anti-depressant activity across BBB and envisaged biomolecular mechanism of action through a proposed pharmacophore model. Journal of microencapsulation 2016; 33(7): 646-655.
47. Li Y, Wu M, Zhang N, Tang C, Jiang P, Liu X et al. Mechanisms of enhanced antiglioma efficacy of polysorbate 80-modified paclitaxel-loaded PLGA nanoparticles by focused ultrasound. Journal of cellular and molecular medicine 2018; 22(9): 4171-4182.
43. Maleklou N, Allameh A, Kazemi B. Preparation, characterization and in vitro-targeted delivery of novel Apolipoprotein E-based nanoparticles to C6 glioma with controlled size and loading efficiency. Journal of drug targeting 2016; 24(4): 348-58.
44. Schilling M, Besselmann Muller M, Strecker J K, Ringetstein E B, Kiefer R. Predominant phagocytic activity of resident microglia over hematogenous macrophages following transient focal cerebral ischemia: an investigation using green fluorescent protein transgenic bone marrow chimeric mice. Experimental neurology 2005; 196(2): 290-7.
45. Neuwelt A, Langsjoen J, Byrd T, Eberhardt S, Mlady G, Baca J et al. Ferumoxytol negatively enhances T2-weighted MRI of pedal osteomyelitis in vivo. Journal of magnetic resonance imaging: JMRI 2017; 45(4): 1241-1245.
46. Neuwelt A, Sidhu N, Hu C A, Milady G, Eberhardt S C, Sillerud L O. Iron-based superparamagnetic nanoparticle contrast agents for MRI of infection and inflammation. AJR. American journal of roentgenology 2015; 204(3): W302-13.

1. A blood-brain barrier-permeable, PEGylated stealth immunomicelle comprising:
(a) a particulate core comprising a mixture of superparamagnetic particles, said core being encapsulated by a plurality of phospholipids comprising at least one pegylated phospholipid and optionally polysorbate 80 and apoE2, a phospholipid comprising conjugation functionalities, and further optionally, a fluorescence-inducing (fluorescent) phospholipid, and/or a cross-linking agent, including a cross-linking phospholipid;
(b) a targeting antibody or peptide or other binding motif which is selected from the group consisting of an anti-Iba-1 or anti-GFAP central nervous system targeting monoclonal or polyclonal antibody which targets Iba-1 or GFAP and which is/are conjugated to said particulate core through a conjugatable phospholipid; and
(c) optionally, a blood-brain barrier-penetration component or ligand which is conjugated to said particulate core through a conjugatable phospholipid which may be the same or different from the conjugatable phospholipid which binds the targeting antibody, peptide or other binding motif.

2. The immunomicelle of claim 1, wherein:
(a) the superparamagnetic particles are superparamagnetic iron platinum particles (SIPP), superparamagnetic iron oxide nanoparticles (SPIONs) or superparamagnetic manganese oxide particles (SMIONs);
(b) the targeting antibody or peptide is selected from the group consisting of (1) anti-Iba-1 or anti-GFAP targeting monoclonal antibody; and
(c) the blood-brain barrier-penetration ligand is Angiopep-1 or polysorbate 80.

3. The immunomicelle of claim 2 wherein:
(a) the pegylated phospholipid is selected from the group consisting of 1,2-distearoyl-sn-glycero-3-phosphoethanolamine-N-[methoxy(polyethylene glycol)-2000] ((DSPE-PEG) or poly(ethylene glycol)-derivatized ceramides (PEG-CER), hydrogenated soy phosphatidylcholine (HSPC), egg phosphatidylcholine (EPC), phosphatidyl ethanolamine (PE), phosphatidyl glycerol (PG), phosphatidyl insitol (PI), monosialogangolioside, spingomyelin (SPM), distearoylphosphatidylcholine (DSPC), dimyristoylphosphatidylcholine (DMPC), and dimyristoylphosphatidylglycerol (DMPG), all of which are pegylated;
(b) the conjugated phospholipid is selected from the group consisting of 1,2-distearoyl-sn-glycero-3-phosphoethanolamine-N-[biotinyl(polyethylene glycol)-2000] ((DSPE-PEG-biotin) 1,2-distearoyl-sn-glycero-3-conjugated phosphoethanolamine-N-[methoxy(polyethylene glycol)-2000] ((DSPE-PEG), conjugated poly(ethylene glycol)-derivatized ceramides (PEG-CER), conjugated hydrogenated soy phosphatidylcholine (HSPC), conjugated egg phosphatidylcholine (EPC), conjugated phosphatidyl ethanolamine (PE), conjugated phosphatidyl glycerol (PG), conjugated phosphatidyl insitol (PI), conjugated monosialogangolioside, conjugated spingomyelin (SPM), conjugated di stearoylphosphatidylcholine (DSPC), conjugated dimyristoylphosphatidylcholine (DMPC), and conjugated dimyristoylphosphatidylglycerol (DMPG);
(c) the immunomicelle optionally comprises a fluorescence-inducing phospholipid which is a phospholipid comprising a fluorescent moiety, wherein the fluorescent moiety is selected from the group consisting of fluoresceins, rhodamines and rhodols, cyanines, phtalocyanines, squairanines, bodipy dyes, pyrene, anthracene, naphthalene, acridine, stilbene, indole, benzindole, oxazole benzoxazole, thiazole, benzothiazole, carbocyanine, carbostyryl, prophyrin, salicylate, anthranilate, azulene, perylene, pyridine, quinoline, borapolyazaindacene, xanthene, oxazine, benzoxazine, carbazine, phenalenone, coumarin, benzofuran, benzphenalenone, rhodamine B, 5-carboxyrhodamine, rhodamine X (ROX), 4,7-dichlororhodamine X (dROX), rhodamine 6G (R6G), 4,7-dichlororhodamine 6G, rhodamine 110 (R110), 4,7-dichlororhodamine 110 (dR110), tetramethyl rhodamine (TAMRA), 4,7-dichlorotetramethylrhodamine (dTAMRA), 4,7-dichlorofluoresceins, 5-carboxyfluorescein (5-FAM) and 6-carboxyfluorescein (6-FAM); and
(d) the cross-linking phospholipid is selected from the group consisting of 2-bis(10,12-tricosadiynoyl)-sn-glycero-3-phosphoethanolamine ((Diyne-PE), 1,2-Dioleoyl-sn-Glycero Phosphocholine (DOPC), 1,2-Dilauroyl-sn-Glycero-3-Phosphocholine (DLPC) and 1-Palmitoyl-2-10,12 Tricosadiynoyl-sn-Glycero-3-Phosphocholine (16:0-23:2 DIYNE PC).

4. The immunomicelle of claim 1, wherein the encapsulated particulate core has an average diameter of (a) between 10 to 150 nm; or (b) 15 nm to 100 nm; or (c) about 25 to about 80 nm.

5. A composition comprising a population of blood-brain barrier-permeable, PEGylated stealth immunomicelles comprising:
(a) a population of PEGylated stealth immunomicelles according to claim 1 comprising a Fe—Pt nanoparticle core wherein the nanoparticles have an average diameter ranging from 10 to 35 nm; and
(b) population of immunomicelles comprising the components which are set forth in Table 1 of Figure S6 hereof, wherein the immunomicelles have an average diameter ranging from around 50 to 80 nm.

6. A pharmaceutical formulation comprising a plurality of the blood-brain barrier-permeable, PEGylated stealth immunomicelles of claim 1 in combination with a pharmaceutically acceptable carrier, additive or excipient.

7. The pharmaceutical formulation of claim 6, wherein said formulation comprises a cardiovascular agent or an agent to control blood pressure.

8. The pharmaceutical formulation of claim 6, wherein said formulation comprises an effective amount of a tissue plasminogen activator.

9. The pharmaceutical formulation of claim 6, wherein the encapsulated particulate cores of each of the immunomicelles are cross-linked.

10. The pharmaceutical formulation of claim 9, wherein the encapsulated particulate cores of each of the immunomicelles are cross-linked by UV-light initiated polymerization.

11. A pharmaceutical formulation comprising a plurality of the blood-brain barrier-permeable, PEGylated stealth immunomicelles of claim 1, wherein the encapsulated particulate cores of each of the immunomicelles are cross-linked.

12. The pharmaceutical formulation of claim 6, wherein the particulate cores of each of the immunomicelles have an average diameter of about 10 to 35 nm.

13. The pharmaceutical composition of claim 12 wherein the particulate cores of each of the immunomicelles have an average diameter of 10 to 50 nm.

14. A method of imaging stroke to determine the stage of microglial macrophage activation in brain tissue of a subject in need comprising administering to the subject a pharmaceutical formulation of any of claims 6-13, subjecting said brain tissue to magnetic resonance imaging and comparing the results obtained with one or more standards.

15. The method of claim 14 wherein the subject undergoes magnetic resonance imaging during or after administration of a pharmaceutical composition.

16. The method of claim 14 wherein the subject undergoes magnetic resonance imaging during or after administration of a treatment for stroke.

17. A method of diagnosing the presence or progression in a subject of stroke comprising:
(a) administering a formulation of claim 13 to the subject;
(b) subjecting the subject to magnetic resonance imaging; and
(c) determining through MM contrast enhancement or determination of iron content in microglia/macrophage of said subject whether the subject suffered from a stroke and the stage of macrophage activation of said stroke.

18. A method of monitoring treatment for stroke in a patient being treated, the method comprising:
(a) administering a formulation of claim 6 to the subject who has suffered a stroke before the commencement of treatment of the subject to determine the level of microglial/macrophage activation;
(b) subjecting the subject to magnetic resonance imaging;
(c) determining the level of macrophage activation and neuroinflammation of the subject;
(d) commencing treatment of the subject;
(e) after a sufficient period of treatment of said subject, administering the same formulation of step (a) to the subject to determine the level of microglial/macrophage activation;
(f) subjecting the subject to magnetic resonance imaging; and
(g) determining through MM contrast enhancement or determination of iron content in microglia of said subject the effect of treatment of said subject.

19. The method of claim 18 wherein said period of treatment is one week.

20. The method of claim 18 wherein said period of treatment is two weeks.

21. The method of claim 18 wherein said period of treatment is three weeks.

22. The method of claim 18 wherein said period of treatment is four weeks.

23. The method of claim 18 wherein said period of treatment is from four to eight weeks.
